(19) United States
(12) Patent Application Publication
(10) Pub. No.: US 2026/0219157 A1
(43) Pub. Date: Jul. 30, 2026

(54) Methods and Systems for Dimensionality Reduction

(71) Applicant: Becton, Dickinson and Company, Franklin Lakes, NJ (US)

(72) Inventors: Josef Spidlen, ASHLAND, OR (US); John Quinn, Yardley, PA (US)

(21) Appl. No.: 19/122,688

(22) PCT Filed: Oct. 27, 2023
(86) PCT No.: PCT/US2023/036138

§ 371 Date: Apr. 18, 2025

Related U.S. Application Data
(60) Provisional application No. 63/421,839, filed on Nov. 2, 2022.

(51) Int. Cl. G01N 15/1429; G01N 15/10; G01N 15/14

(52) U.S. Cl. CPC G01N 15/1429; G01N 2015/1006; G01N 2015/1402

(57) ABSTRACT

Computer-implemented methods of dimensionality reduction are provided. Methods of interest include receiving a secondary dataset comprising data points collected from a secondary sample, and calculating a second dimensionality reduction for the secondary dataset based on a first dimensionality reduction of a reference dataset comprising data points collected from a reference sample. The calculated second dimensionality is compatible with the dimensionally reduced reference dataset. Systems and non-transitory computer readable storage media configured to perform the methods of the invention are also provided.

### CROSS-REFERENCE TO RELATED APPLICATIONS

Pursuant to 35 U.S.C. § 119 (e), this application claims priority to the filing date of U.S. Provisional Patent Application Ser. No. 63/421,839 filed Nov. 2, 2022; the disclosure of which application is incorporated herein by reference in its entirety.

### INTRODUCTION

Flow cytometry is a technique used to characterize and often times sort biological material, such as cells of a blood sample or particles of interest in another type of biological or chemical sample. A flow cytometer typically includes a sample reservoir for receiving a fluid sample, such as a blood sample, and a sheath reservoir containing a sheath fluid. The flow cytometer transports the particles (including cells) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of detectors, one for each of the scatter parameters to be measured, and one or more for each of the distinct dyes to be detected are included in the analyzer. For example, some embodiments include spectral configurations where more than one sensor or detector is used per dye. The data obtained comprise the signals measured for each of the light scatter detectors and the fluorescence emissions.

Flow cytometers may further comprise means for recording the measured data and analyzing the data. For example, data storage and analysis may be carried out using a computer connected to the detection electronics. For example, the data can be stored in tabular form, where each row corresponds to data for one particle, and the columns correspond to each of the measured features. The use of standard file formats, such as an “FCS” file format, for storing data from a particle analyzer facilitates analyzing data using separate programs and/or machines. Using current analysis methods, the data typically are displayed in 1-dimensional histograms or 2-dimensional (2D) plots for ease of visualization, but other methods may be used to visualize multidimensional data.

The parameters measured using, for example, a flow cytometer typically include light at the excitation wavelength scattered by the particle in a narrow angle along a mostly forward direction, referred to as forward scatter (FSC), the excitation light that is scattered by the particle in an orthogonal direction to the excitation laser, referred to as side scatter (SSC), and the light emitted from fluorescent molecules in one or more detectors that measure signal over a range of spectral wavelengths, or by the fluorescent dye that is primarily detected in that specific detector or array of detectors. Different cell types can be identified by their light scatter characteristics and fluorescence emissions resulting from labeling various cell proteins or other constituents with fluorescent dye-labeled antibodies or other fluorescent probes.

Common high-dimensional analysis workflows include creating, or deriving, a set of parameters that represent the cells in a low-dimensional graph by creating a smaller set of parameters (most commonly two) that attempt to summarize’ information from all other parameters. This is known as dimensionality reduction (dim redux). A typical dimensionality reduction workflow assumes that users concatenate all input samples and then run dimensionality reduction on a single concatenated data set. Conventionally, flow cytometry users performing dimensionality reduction, such as tSNE, UMAP, EmbedSOM, TriMap, PacMAP etc. are forced concatenate input samples up front as there is not a generic solution that would allow them to calculate embedding of a sample based on previously calculated embeddings of different samples. For example, FIG. 1 depicts a typical dimensionality reduction workflow in flow cytometry. As shown in FIG. 1, datasets 101a-101c (e.g., in the form of .fcs files) from different samples are concatenated into a single file 102, based on which new parameters 103 are derived. A common follow up step is to use a clustering algorithm to partition the data into like and unalike groups of cells that generally align with phenotypes, and store the cluster membership number as a derived parameter.

These aforementioned approaches are generally non-deterministic; they will produce different results if run multiple times. Cells of the same or similar phenotypes will group together when the algorithm is run, but they will not always be oriented the same, making direct comparison difficult, nor will the same phenotype always be assigned the same cluster number. Traditionally, all of the data to be compared need to be included in the parameter creation step, meaning that it must all be processed at one time. If additional data sets are generated, the entire process needs to be restarted.

### SUMMARY

The present inventors have realized that conventional methods of dimensionality reduction are not practical because they require all data to be collected up front, and also require computing dimensionality reduction on large input files. This leads to challenges with computing resources and calculation time. Furthermore, adding new data to an analysis requires redoing the entire analysis, thereby exacerbating inefficiencies. The concatenation step may also lose connection to metadata that then needs to be tracked manually. In addition, it was discovered that there is no generic solution that would allow users to calculate an embedding of a sample based on previously calculated embeddings of different samples as opposed to embedding new samples into an existing dimensionality reduction. Accordingly, an algorithmic technique where dimensionality reduction is calculated for a new sample based on an existing dimensionality reduction of a different (i.e., reference) sample is desirable. The methods, systems and computer readable media of the present invention satisfy this desire.

Aspects of the invention include computer-implemented methods of dimensionality reduction. Methods of interest include receiving a secondary dataset comprising data points collected from a secondary sample, and calculating a second dimensionality reduction for the secondary dataset based on a first dimensionality reduction of a reference dataset comprising data points collected from a reference sample. The second dimensionality reduction calculated in the subject methods is compatible with the dimensionally reduced reference dataset. In some cases, methods include determining data points collected from the reference sample that are closest to each data point collected from the secondary sample with respect to one or more given data parameters. For example, calculating the second dimensionality reduction may include calculating k nearest neighbors within the reference dataset for each data point collected from the secondary sample. The value for k may vary, and can range from 1 to m, where m is half of the number of data points in the reference sample. In some cases, k ranges from 2 to 5. In embodiments, the method comprises calculating the k nearest neighbors using a vantage-point tree, a k-dimensional tree, ball tree, cover tree, locality-sensitive hashing, hierarchical navigable small world, approximate nearest neighbors with random projection trees, GPU-based KNN search, or a brute force KNN search. In embodiments, methods include calculating a distance (e.g., Manhattan distance, Euclidean distance, Chebyshev distance, Minkowski distance, cosine distance) between each data point collected from the secondary sample and each nearest neighbor of the k nearest neighbors. In certain versions, methods include calculating the second dimensionality reduction based on a weighted average of positions of the k nearest neighbors. In other cases, such as where categorical parameters are derived, methods include calculating the second dimensionality reduction based on a weighted majority-voting approach. Methods according to some embodiments include calculating the weighted average using a weight obtained for each nearest neighbor of the k nearest neighbors based on the distance. In some cases, the secondary dataset comprises data points associated with a plurality of parameters that are matched to the reference dataset. Methods according to some embodiments include creating derived parameters for the secondary dataset based on parameters (e.g., fluorescence parameters, scatter parameters, imaging parameters or categorical parameters) of the dimensionally reduced reference dataset. In some cases, the derived parameters are fluorescence parameters. In select versions, creating the derived parameters for the secondary dataset comprises linear interpolation.

Methods according to some embodiments, include calculating quality scores designed to indicate whether data is sufficiently stable over time. In some such cases, methods include calculating an input quality score indicating an extent to which each data point collected from the secondary sample is related to the data points within the reference dataset. Calculating an input quality score may include, for example, obtaining a normalized average distance of each data point collected from the secondary sample to the k nearest neighbors. In additional cases, methods include calculating an output quality score measuring the separation of the k nearest neighbors in the dimensionally reduced first dataset. Calculating an output quality score may include, for example, obtaining a normalized average distance of each of the k nearest neighbors within the dimensionally reduced reference dataset.

In some cases, the reference and secondary datasets are comprised of flow cytometer data. In select versions, the reference dataset is a uniformly down-sampled reference dataset or a density-based down-sampled reference dataset. In certain implementations, the method comprises receiving the reference dataset, and performing the first dimensionality reduction. Methods according to some embodiments include transforming the reference dataset and the secondary dataset, e.g., with a linear function, logarithmic function, a hyperbolic arcsine function, or a biexponential function. In some cases, the method comprises calculating a dimensionality reduction for a plurality of secondary datasets comprising data points collected from a plurality of secondary samples.

Aspects of the invention additionally include systems and non-transitory computer readable storage media configured to perform the subject methods (e.g., as set forth above and herein). For example, aspects of the invention include a processor comprising memory operably coupled to the processor, wherein the memory comprises instructions stored thereon, which when executed by the processor, cause the processor to receive a secondary dataset comprising data points collected from a secondary sample, and calculate a second dimensionality reduction for the secondary dataset based on a first dimensionality reduction of a reference dataset comprising data points collected from a reference sample. The second dimensionality reduction calculated by the processor is compatible with the dimensionally reduced reference dataset. Similarly, aspects of the invention include a non-transitory computer readable storage medium comprising instructions stored thereon for dimensionality reduction by a method comprising receiving a secondary dataset comprising data points collected from a secondary sample, and calculating a second dimensionality reduction for the secondary dataset based on a first dimensionality reduction of a reference dataset comprising data points collected from a reference sample.

### BRIEF DESCRIPTION OF THE FIGURES

The invention may be best understood from the following detailed description when read in conjunction with the accompanying drawings. Included in the drawings are the following figures:

FIG. 1 depicts a conventional dimensionality reduction workflow in flow cytometry.

FIG. 2 depicts a dimensionality reduction workflow according to certain embodiments of the invention.

FIG. 3 presents a conceptual illustration of a dimensionality reduction method according to embodiments of the invention.

FIG. 4 presents a dimensionality reduction workflow according to certain embodiments of the invention.

FIG. 5 depicts an exemplary graphical user interface for use during dimensionality reduction according to certain embodiments of the invention.

FIG. 6 depicts a functional block diagram of a flow cytometer according to certain embodiments.

FIG. 7 depicts a control system according to certain embodiments of the invention.

FIG. 8A-8B depict schematic drawings of a particle sorter system according to certain embodiments.

FIG. 9 depicts an image-enabled particle sorter according to certain embodiments.

FIG. 10 depicts a block diagram of a computing system according to certain embodiments.

FIG. 11A-11B depict an exemplary dimensionality reduction on a first sample (FIG. 11A) and a second sample (FIG. 11B).

FIG. 12A-12B depict an exemplary dimensionality reduction showing embeddings using overlays.

FIG. 13A-13B depict an exemplary dimensionality reduction showing embeddings using overlays.

FIG. 14A-14B illustrate how input and output quality score can be leveraged to gate events with high quality embedding results.

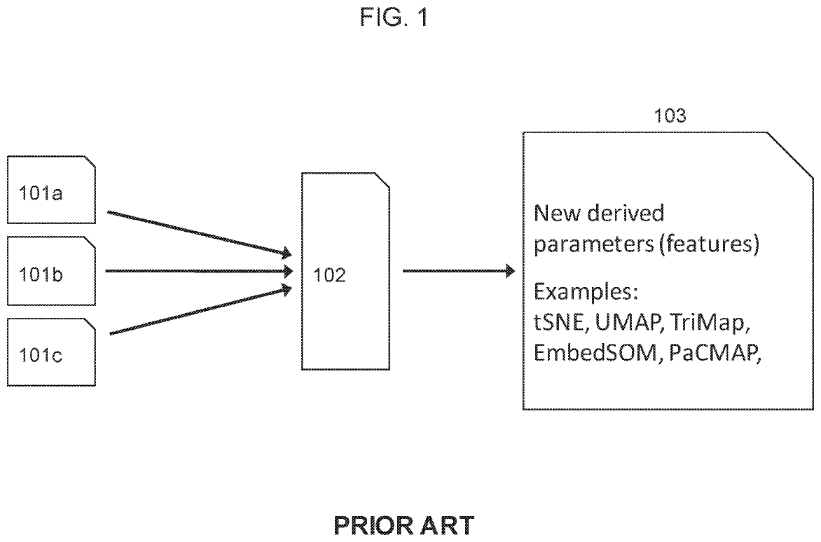

### DETAILED DESCRIPTION

Computer-implemented methods of dimensionality reduction are provided. Methods of interest include receiving a secondary dataset comprising data points collected from a secondary sample, and calculating a second dimensionality reduction for the secondary dataset based on a first dimensionality reduction of a reference dataset comprising data points collected from a reference sample. The calculated second dimensionality is compatible with the dimensionally reduced reference dataset. Systems and non-transitory computer readable storage media configured to perform the methods of the invention are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Computer-Implemented Methods of Dimensionality Reduction

As discussed above, aspects of the invention include computer-implemented methods of dimensionality reduction. The term “dimensionality reduction” is referred to in its conventional sense to describe the conversion of data from a high-dimensional space to a comparatively lower dimensional space. Generally and/or ideally, the dimensionally reduced data (i.e., data in low-dimensional space) retains properties of the data in high-dimensional space such that said properties are represented in the dimensionally reduced data in some form. The methods of the invention enhance the conversion of data from a high-dimensional space to a comparatively lower dimensional space by, e.g., improving data quality and providing a means by which users may be notified if data quality is insufficient. In addition, methods according to some embodiments of the invention improve computational efficiency by 5% or more, such as 10% or more, such as 15% or more, such as 20% or more, such as 25% or more and including 30% or more.

Methods of the invention involve calculating a second dimensionality reduction for a secondary dataset based on a first dimensionality reduction of a reference dataset comprising data points collected from a reference sample. The second dimensionality reduction calculated by the subject methods is compatible with the dimensionally reduced reference dataset. By “compatible”, it is meant that the second dimensionality reduction enables meaningful comparison between the secondary dataset and the dimensionally reduced reference dataset. As discussed above in the Introduction section, prior approaches to dimensionality reduction are non-deterministic, i.e., they will produce different results if run multiple times. In contrast, the compatibility of the second dimensionality reduction with the dimensionally reduced reference dataset means that data clusters within a dimensionally reduced dataset generated by the second dimensionality reduction calculated by the subject methods will have identifiable characteristics that will enable assessment relative to corresponding clusters in the dimensionally reduced reference dataset, even if the secondary dimensionality reduction is run multiple times. For example, cells of the same or similar phenotypes grouped together in clusters within a dimensionally reduced dataset generated by the second dimensionality reduction calculated by the subject methods may have the same or similar orientations relative to corresponding clusters in the dimensionally reduced reference dataset. In addition, cells of the same or similar phenotypes grouped together in clusters within a dimensionally reduced dataset generated by the second dimensionality reduction calculated by the subject methods may have the same or similar cluster numbers relative to corresponding clusters in the dimensionally reduced reference dataset. In some cases, multiple iterations of the secondary dimensionality reduction of the invention will result in dimensionally reduced data that differs between iterations with respect to cluster-identifying characteristics (e.g., cluster number, orientation, etc.) by 10% or less, such as 9% or less, such as 8% or less, such as 7% or less, such as 6% or less, such as 5% or less, such as 4% or less such as 3% or less, such as 2% or less, and including 1% or less.

The dimensionality reduction of the invention may take any suitable form. In some embodiments, dimensionality reduction is performed by a t-Distributed Stochastic Neighbor Embedding (t-SNE) algorithm. The t-SNE algorithm is described in Laurens van der Maaten & Geoffrey Hinton. Learning Research. (2008); herein incorporated by reference. In some embodiments, dimensionality reduction is performed by a Uniform Manifold Approximation and Projection (UMAP) algorithm. The UMAP algorithm is described in McInnes et al. ARXIV. (2018); herein incorporated by reference. In some embodiments, dimensionality reduction is performed by a TriMap algorithm. The TriMap algorithm is described in Ehsan Amid & Manfred K. Warmuth. ARXIV. (2019); herein incorporated by reference. In some embodiments, dimensionality reduction is performed by the EmbedSOM algorithm. The EmbedSOM algorithm is described in, e.g., Kratochvil et al. bioRxiv. (2018), incorporated by reference herein. In some cases, the dimensionality reduction is performed by the PacMAP algorithm. The PacMAP algorithm is described in, e.g., Wang et al. The Journal of Machine Learning Research. (2021) 22(1): 9129-9201, the disclosure of which is incorporated by reference herein. In some cases, the first and secondary dimensionality reductions are selected from tSNE, UMAP, EmbedSOM, TriMap, and PacMAP, although any dimensionality reduction that currently exists or has yet to be invented may be adapted for use herein.

Methods of the invention include receiving a secondary dataset comprising data points collected from a secondary sample. The secondary dataset may be received from any convenient source. In some embodiments, the secondary dataset is received from a flow cytometer. In such embodiments, the secondary dataset is considered flow cytometer data and is received from any suitable flow cytometer, including but not limited to those described herein. Exemplary flow cytometers include BD Biosciences FACSCanto™ flow cytometer, BD Biosciences FACSCanto™ II flow cytometer, BD Accuri™ flow cytometer, BD Accuri™ C6 Plus flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer, BD Biosciences LSRFortessa™ flow cytometer, BD Biosciences LSRFortessa™ X-20 flow cytometer, BD Biosciences FACSPresto™ flow cytometer, BD Biosciences FACSVia™ flow cytometer and BD Biosciences FACSCalibur™ cell sorter, a BD Biosciences FACSCount™ cell sorter, BD Biosciences FACSLyric™ cell sorter, BD Biosciences Via™ cell sorter, BD Biosciences Influx™ cell sorter, BD Biosciences Jazz™ cell sorter, BD Biosciences Aria™ cell sorter, BD Biosciences FACSAria™ II cell sorter, BD Biosciences FACSAria™ III cell sorter, BD Biosciences FACSAria™ Fusion cell sorter and BD Biosciences FACSMelody™ cell sorter, BD Biosciences FACSymphony™ S6 cell sorter or the like. In some cases, the secondary dataset is received from a database. In such cases, the database may be hosted locally, e.g., on any of a variety of known or future memory storage devices. Examples include any commonly available random access memory (RAM), magnetic medium such as a resident hard disk or tape, an optical medium such as a read and write compact disc, flash memory devices, or other memory storage device. Alternatively, the database may be an internet database. Exemplary internet databases include, but are not limited to, the FlowRepository database (flowrepository(dot)org). In some cases where the secondary dataset includes flow cytometer data, the data points within the dataset may be considered “events”. In some embodiments, the secondary dataset is provided as a .fcs file.

In some cases, methods include receiving a plurality of secondary datasets. For example, in some cases, methods include receiving a number of secondary datasets ranging from 2 to 50, such as 2 to 25 and including 2 to 10. In some instances, methods include receiving 2 or more secondary datasets, such as 3 or more secondary datasets, such as 4 or more secondary datasets, such as 5 or more secondary datasets, such as 6 or more secondary datasets, such as 7 or more secondary datasets, such as 8 or more secondary datasets, such as 9 or more secondary datasets, and including 10 or more secondary datasets. In some such versions, methods include calculating a dimensionality reduction for a plurality of secondary datasets comprising data points collected from a plurality of secondary samples.

Additionally, methods may include receiving a reference dataset, and performing the first dimensionality reduction. In such embodiments, the first dimensionality reduction may be conducted according to any suitable method of dimensionality reduction. For example, in some embodiments, the first dimensionality reduction is selected from tSNE, UMAP, EmbedSOM, TriMap, and PacMAP. Generally, the same type of dimensionality reduction is employed for the secondary dataset, with adjustments made as described below. Alternatively, the reference dataset and first dimensionality reduction may be stored on a memory operably coupled to a processor configured to carry out the subject methods. In some cases, the reference dataset is a uniformly down-sampled reference dataset or a density-based down-sampled reference dataset. The reference dataset may be received from any convenient source, including but not limited to those discussed above with respect to the secondary dataset(s). In some instances, methods include receiving a plurality of reference datasets. For example, in some cases, methods include receiving a number of reference datasets ranging from 2 to 50, such as 2 to 25, and including 2 to 10.

In some embodiments, calculating the second dimensionality reduction comprises determining data points collected from the reference sample that are closest to each data point collected from the secondary sample with respect to given data parameters. The “parameters” refer to characteristics of an analyte being measured. For example, in an embodiment where the reference and secondary datasets include flow cytometer data, parameters may include, e.g., forward scatter (FSC), side scatter (SSC), CD3, CD4, CD8, CD25 and the like. In some cases, parameters are fluorescent parameters, i.e., the characteristic of interest is measured by way of its association with a fluorescent molecule (e.g., fluorochrome). The parameters discussed herein may also be embedding parameters, e.g., parameters related to the type of dimensionality reduction being performed (e.g., t-SNE, UMAP, EmbedSOM, PacMAP, etc.). In select cases, the determination of data points collected from the reference sample that are closest to each data point collected from the secondary sample is conducted with respect to parameters that are known to exist in both the reference dataset and secondary dataset. In some cases, the secondary dataset comprises data points associated with a plurality of parameters that are matched to the reference dataset. In other words, the parameters are common to the two datasets. As an example, if the dimensionally reduced reference dataset has a parameter set of {CD3, CD4, CD8, CD25, tSNE1, tSNE2}, and the secondary dataset has a parameter set of {CD3, CD4, CD8, and CD25}, the four common parameters {CD3, CD4, CD8, and CD25} may be used for the calculation.

In some cases, calculating the second dimensionality reduction comprises a proximity search configured to find a point or points within a set that is/are closest to one or more data points collected from the secondary sample. For example, in some cases, methods include calculating a nearest neighbor for each data point collected from the secondary sample. The nearest neighbor problem may be defined as finding a closest point in S to q, where S is a set of points in a space M and q E M. In select instances, methods include calculating k nearest neighbors within the reference dataset for each data point collected from the secondary sample, where k is a positive integer. While the value for k may vary, in some embodiments k ranges from 1 to m, where m is equal to half of the number of data points in the reference sample. In some cases, k ranges from 2 to 5. In certain cases, k is 1 or more, such as 2 or more, such as 3 or more, such as 4 or more, such as 5 or more, such as 6 or more, such as 7 or more, such as 8 or more, such as 9 or more, and including 10 or more. The method by which the k nearest neighbors is calculated may vary. In some cases, the method comprises calculating the k nearest neighbors using a vantage-point tree, a k-dimensional tree, ball tree, cover tree, locality-sensitive hashing, hierarchical navigable small world, approximate nearest neighbors with random projection trees, GPU-based KNN search, or a brute force KNN search. In some cases, the method comprises calculating the k nearest neighbors using a vantage-point tree. Vantage-point trees are described in, e.g., Yianilos, Peter N. Soda. (1993) 93(194):311-21, incorporated by reference herein. In select instances, the method comprises calculating the k nearest neighbors using a k-dimensional tree (k-d tree). k-dimensional trees are described in, e.g., Bentley, J. L. Comm. ACM. (1975) 18(9):509 517, incorporated by reference herein. In select instances, the method comprises calculating the k nearest neighbors using a ball tree (metric tree). Ball trees are described in, e.g., Omohundro, S. M. Five balltree construction algorithms. (1989), incorporated by reference herein. In some cases, the method comprises calculating the k nearest neighbors using locally sensitive hashing. Locally sensitive hashing is described in, e.g., Pauleve et al. Pattern recognition letters. (2010) 31(11): 1348-1358, incorporated by reference herein. In some cases, the method comprises calculating the k nearest neighbors using hierarchical navigable small world (HNSW). Hierarchical navigable small world is described in, e.g., Malkov et al. IEEE transactions on pattern analysis and machine intelligence. (2018) 42(4): 824-836, incorporated by reference herein. In some cases, the method comprises calculating the k nearest neighbors using approximate nearest neighbors with random projection trees. Such trees are described in, e.g., Hyvönen et al. In 2016 IEEE International Conference on Big Data (Big Data), pp. 881-888, incorporated by reference herein. In some cases, the method comprises calculating the k nearest neighbors using a GPU-based KNN search. GPU-based KNN searches are described in, e.g., Garcia et al. In 2010 IEEE International Conference on Image Processing, pp. 3757-3760, incorporated by reference herein. In some cases, the method comprises performing a brute force KNN search.

Methods according to some embodiments additionally include calculating a distance between each data point collected from the secondary sample and each nearest neighbor of the k nearest neighbors. The distance calculated may vary. In some cases, the distance is a Euclidean distance, i.e., length of a line segment between the two points. In other cases, the distance is a Manhattan distance in which the distance between two points is the sum of the absolute differences of their Cartesian coordinates. In still other cases, the distance is a Chebyshev distance, i.e., the greatest of the differences between two vectors along any coordinate dimension. In yet other cases, the distance is a Minkowski distance, i.e., a generalization of the Euclidian distance and the Manhattan distance. In still other cases, the distance is a cosine distance, i.e., the complement of cosine similarity. In certain instances, the distance is selected from Euclidean distance, Manhattan distance, Chebyshev distance, Minkowski distance and cosine distance.

In embodiments, methods include calculating the second dimensionality reduction based on a weighted average of positions of the k nearest neighbors. In other words, methods include taking a weighted average of the derived parameters in the reference dataset. Put another way, the position of each data point in the secondary dataset in dimensionally reduced space is defined by a weighted position of its nearest neighbors from higher dimensional space in the dimensionally reduced space. The weighted average may be calculated using a weight obtained for each nearest neighbor of the k nearest neighbors based on the distance (e.g., Euclidean distance, Manhattan distance, Chebyshev distance, Minkowski distance and cosine distance. as desired). In other words, the nearest neighbors are weighted differently in the average depending on their distance. Closer neighbors have a larger weight. Weights for use in the weighted average may vary. In some cases, the method comprises obtaining the weight as follows:

$${w}_{i} = \frac{1 + a}{1 + {d}_{i} + b}$$

where wᵢ is the weight, dᵢ is the distance, and a and b are real numbers. In additional cases, the method comprises obtaining the weight as follows:

$${w}_{i} = f ( \frac{1 + a}{1 + {d}_{i} + b} )$$

where f is any monotonic entirely non-decreasing function, wᵢ is the weight, dᵢ is the distance, and a and b are real numbers. In still further cases, the method comprises obtaining the weight as follows:

$${w}_{i} = \frac{1}{1 + {d}_{i}}$$

where wᵢ is the weight, and dᵢ is the distance. As shown in the formulas above, weights increase as distances decrease.

In some embodiments, methods include creating derived parameters for the secondary dataset based on parameters of the dimensionally reduced reference dataset. In such cases, the parameters are selected from fluorescence parameters, scatter parameters, imaging parameters or categorical parameters. In certain implementations, the derived parameters are fluorescence parameters. For example, the computer-implemented methods of the invention may be used to create “virtual tubes”. In other words, assuming that a backbone of shared parameters exists between a reference dataset/reference sample and a secondary dataset/secondary sample, the value of fluorescence parameters that do not exist for the secondary dataset can be estimated based on those parameters in the reference dataset. This would allow users to create “virtual tubes” to visualize co-expression patterns of markers that aren't present on any single tube (i.e., dataset) together. In additional implementations, the derived parameters are scatter parameters. In still further implementations, the derived parameters are imaging parameters. Where the parameters are fluorescence parameters, scatter parameters, or imaging parameters, said parameters may be calculated by the weighted average discussed above. In some embodiments, the parameters are categorical parameters, such as clustering parameters. In such embodiments, the calculation is performed by weighted majority-voting. For example, if a data point's seven nearest neighbors belonged to clusters {1,1,1,1,2,1,3}, this data point would be assigned to cluster 1 as the majority of the neighbors belong to this cluster. For data points whose nearest neighbors lack a majority cluster, said data points may be assigned to cluster −1, i.e., an outlier cluster. Alternatively, methods may include placing all data points in a cluster, e.g., if an experiment requires that all cells be classified. In this case, methods include assigning data points to the same cluster as the single nearest neighbor.

In some cases, creating the derived parameters for the secondary dataset comprises interpolation. In other words, the parameters for the data points within the secondary dataset are interpolated based on the known data points within the reference dataset and dimensionally reduced reference dataset. In some instances, the interpolation is nearest-neighbor interpolation. In other instances, the interpolation is linear interpolation. This process may be used, e.g., for continuous variables such as dimensionality reduction parameters.

Methods according to some embodiments include calculating quality scores designed to indicate whether data is sufficiently stable over time. In some cases, a user may review the one or more quality scores to determine the quality of the second dimensionality reduction. For example, the quality scores may denote an extent to which the reference and secondary dataset(s) may be meaningfully compared. The quality scores may, in some cases, indicate the accuracy with which parameters are derived for the secondary dataset. In some embodiments, methods include calculating an input quality score indicating an extent to which each data point collected from the secondary sample is related to the data points within the reference dataset. In some cases, input quality for a given cell is the average of distance of the nearest neighbors to the cell, normalized by the number of dimensions used. In simple terms, the smaller this number is, the better the nearest neighbors represent the target cell. Normalization may differ depending on the distance metric employed. In embodiments where the distance is a Manhattan distances, normalization is performed by dividing by the number of dimensions used in the calculation. In embodiments where the distance is a Euclidian distance, normalization may be performed by dividing by the square root of the number of dimensions used in the calculation. In embodiments where the distance is a Chebyshev distance, no normalization is required because the score is already normalized. Where normalization is employed, said normalization may derive a score that is reasonably independent of the number of dimensions and distance metric used.

In additional embodiments, methods include calculating an output quality score measuring the separation of the k nearest neighbors in the dimensionally reduced first dataset. In some cases, calculating the output quality score comprises obtaining a normalized average distance of each of the k nearest neighbors within the dimensionally reduced reference dataset. In other words, output quality for a given cell is the average normalized distance between the nearest neighbor cells in the embedded parameter space. Smaller numbers are better. A large number could indicate that nearest neighbors in the measured parameter space have been significantly separated in the dimensionally reduced space (e.g., tSNE splitting homogeneous populations into separate islands). Normalization may be conducted as described above with respect to the input quality score.

Methods may additionally include transforming the reference dataset and the secondary dataset. Specifically, methods according to some embodiments include transforming parameters in the reference dataset and parameters in the secondary dataset. In some embodiments, the method comprises transforming parameters in the reference dataset and parameters in the secondary dataset with a linear function, logarithmic function, a hyperbolic arcsine function, or a biexponential function. In some embodiments, the method comprises transforming parameters in the reference dataset and parameters in the secondary dataset with a linear function. In some embodiments, the method comprises transforming parameters in the reference dataset and parameters in the secondary dataset with a logarithmic function. In some embodiments, the method comprises transforming parameters in the reference dataset and parameters in the secondary dataset with a hyperbolic arcsine function. In some such embodiments, the hyperbolic arcsine function is performed with a co-factor of 150. In some embodiments, the method comprises transforming parameters in the reference dataset and parameters in the secondary dataset with a biexponential function.

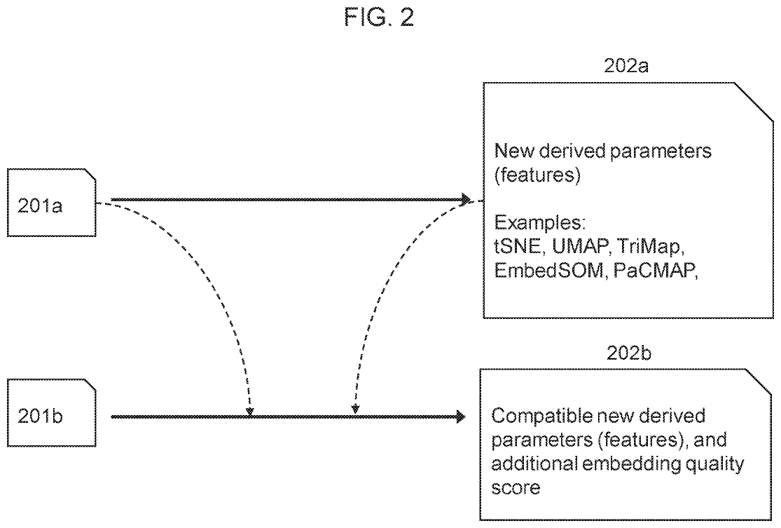

FIG. 2 presents an approach to dimensionality reduction according to embodiments of the invention. A first dimensionality reduction is performed on reference dataset 201a comprising data points collected from a reference sample. This results in a dimensionally reduced reference dataset comprising derived parameters 202a. The reference dataset 201a as well as the derived parameters 202a are used to calculate a secondary dimensionality reduction for secondary dataset 201b, which results in parameters 202b that are compatible with the derived parameters 202a.

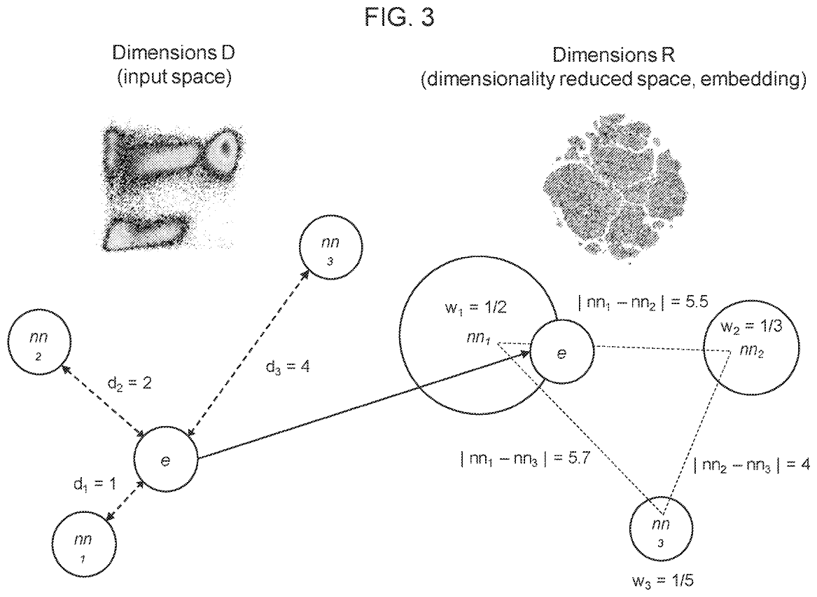

FIG. 3 presents a conceptual illustration of a dimensionality reduction method according to embodiments of the invention. First, a dimensionality reduction R from dimensions D is performed on a reference dataset from a reference sample. Subsequently, methods include calculating a dimensionality reduction R for a secondary dataset that is compatible with the dimensionally reduced reference dataset. This involves, for each data point (event e) in the secondary dataset: calculating NN as k nearest-neighbors of e within the reference dataset using dimensions D. In the example of FIG. 3, nearest neighbors nn₁, nn₂ and nn₃ are depicted. Methods also include, for each nearest-neighbor nnᵢ of NN, calculating distance dᵢ as distance from nnᵢ to e. In the example of FIG. 3, distance d₁ between nn₁ and e is 1, distance d₂ between nn₂ and e is 2, and distance d₃ between nn₃ and e is 4.

Next, methods include calculating input quality score for e as the average of dᵢ normalized by the number of dimensions in D. In other words, input quality is the normalized average distance of e to its nearest neighbors in D, the closer the neighbors, the better, i.e., small numbers are better. In this example, the input quality score is calculated by ((1+2+4)/3)/ND, where ND varies depending on the type of distance metric used. Where the distance is a Manhattan distance, ND=∥D∥. Where the distance is a Euclidian distance, ND=sqrt(∥D∥). Where the distance is a Chebyshev distance, ND=1. Subsequently, methods include calculating an output quality score for e as the average normalized distance between nnᵢ and nnⱼ where i!=j in R. In other words, output quality is the normalized average distance of the nearest neighbors from each other in R, the closer the neighbors, the better, i.e., small numbers are better. In this example, distances are calculated as follows: |nn₁−nn₂|=5.5, |nn₂−nn₃|=4, and |nn₁−nn₃|=5.7. The output quality score is calculated by ((5.5+4+5.7)/3)/NR, where NR varies depending on the type of distance metric used. Where the distance is a Manhattan distance, NR=∥R∥. Where the distance is a Euclidian distance, NR=sqrt(∥R∥). Where the distance is a Chebyshev distance, NR=1.

The data points in the reference dataset identified as nearest neighbors are then used to create derived parameters for the data points in the secondary dataset using, e.g., linear interpolation for continuous variables such as dimensionality reduction parameters. This involves taking a weighted average of the derived parameters in the reference data set. Weighting is performed by the proximity of the given cell to each nearest neighbor. In other words, for each dᵢ calculating weight wᵢ as wᵢ=1/(1+dᵢ). In the example of FIG. 3, weight w₁ for nn₁ is ½, weight w₂ for nn₂ is ⅓, and weight w₃ for nn₃ is ⅕. Subsequently, for each reduced dimension r of R using wᵢ as weights, methods include calculating an r value of e as weighted average of r values of the nearest neighbors NN.

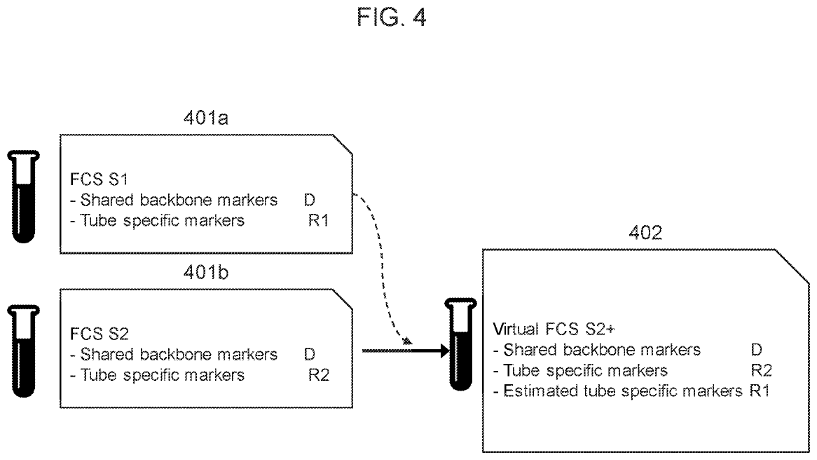

FIG. 4 presents a dimensionality reduction workflow involving derived fluorescence parameters according to certain embodiments of the invention. As shown in FIG. 4, reference dataset 401a and secondary dataset 401b possess a shared backbone of parameters/markers D. Reference dataset 401a has parameters/markers R1 specific to that dataset for reference sample S1, and secondary dataset 401b has parameters/markers R2 specific to that dataset for secondary sample S2. Methods of the invention (e.g., described above) may be used to create a “virtual tube” 402 for the secondary sample where tube specific markers R1 are estimated in the secondary dataset.

In some cases, methods include receiving input from a user. The input may relate to, e.g., which parameters are derived for the secondary dataset and/or the quality score(s) that should be calculated. In some cases, users select parameters that are known to be common to the two datasets, i.e., parameters in the secondary dataset that are matched to the reference dataset. In certain cases, methods include inputting a value for k for the k-nearest neighbors search discussed above. In select embodiments, methods include selecting a distance metric to be used in the calculations (e.g., Manhattan distance, Euclidian distance, Chebyshev distance, Minkowski distance, or cosine distance). In embodiments, methods include selecting a number of datapoints in the reference dataset that are used in the calculation. In additional cases, a user may select whether an input quality score is calculated. In still additional cases, a user may select whether an output quality score is calculated. These selections may be achieved, e.g., via an input manager. In some cases, the input manager is operatively coupled to a graphical user interface where the input is entered. In certain instances, input is entered on an internet website menu interface (e.g., at a remote location) and communicated to the input manager, over the internet or a local area network. In some embodiments, the input manager is operatively coupled to one or more searchable databases (e.g., catalogs) of parameters. In certain instances, the input manager includes a database of parameters. All or part of each database parameters may be displayed on the graphical user interface, such as in a list, drop-down menu or other configuration (e.g., tiles). For example, the graphical user interface may display a list of parameters simultaneously (i.e., on a single screen) or may contain one or more drop-down menus. In some cases, the graphical user interface comprises a plurality of drop down menus: a first menu for selecting parameters in a reference dataset and a second menu for selecting target parameters in the secondary dataset to be derived and embedded. In other embodiments, information may be input into appropriate text fields, selecting check boxes, selecting one or more items from a drop-down menu, or by using a combination thereof. Methods of the invention may include using selected parameters for the methods of the invention.

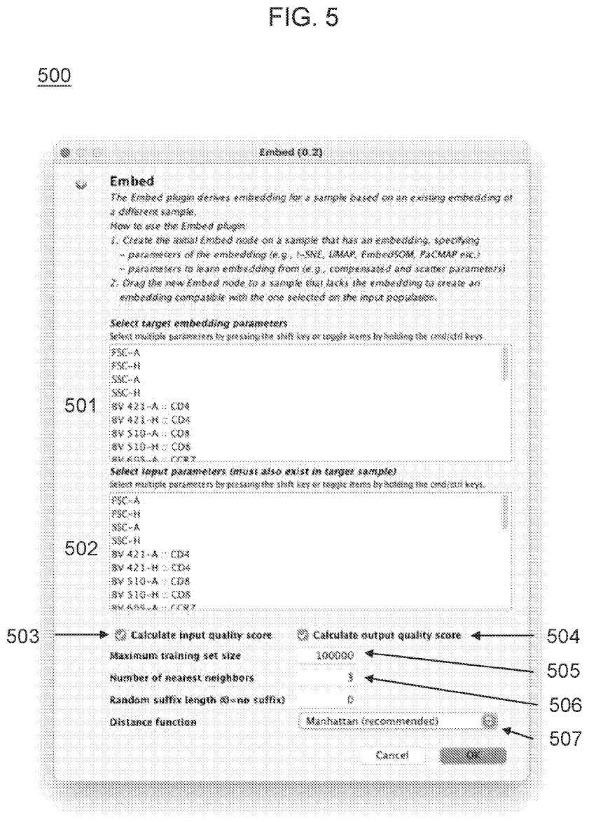

FIG. 5 depicts an exemplary graphical user interface 500 for receiving input from a user. Graphical user interface 500 includes menu 502 for selecting parameters that are common between a reference dataset and a secondary dataset, and menu 503 for selecting parameters that are to be derived/embedded in the secondary dataset during dimensionality reduction. In the example of FIG. 5, check boxes 503 and 504 are provided for indicating whether an input quality score and output quality score should be calculated, respectively. Graphical user interface 500 additionally includes a text field 505 for indicating a maximum size for the reference dataset. Maximum training size caps the search area for detecting nearest neighbors, essentially downsampling to the entered number before finding nearest neighbors. The user may also indicate in text field 506 a number of nearest neighbors (i.e., k) used for calculating k nearest neighbors within the reference dataset for each data point collected from the secondary sample. Graphical user interface 500 also includes drop down menu 507 for selecting a distance metric.

As discussed above, the reference and secondary datasets may in some embodiments include flow cytometer data. In certain instances, the flow cytometer data is fluorescent flow cytometer data. By “fluorescent flow cytometer data” it is meant information regarding parameters of a sample (e.g., cells, particles) in a flow cell that is collected by any number of fluorescent light detectors in a particle analyzer. In embodiments, fluorescent flow cytometer data includes signals from a plurality of different fluorochromes, such as, for instance, ranging from 2 to 40 different fluorochromes, including 3 to 30 different fluorochromes, such as 3 to 20 different fluorochromes, and in some instances including 3 to 5 different fluorochromes. In some embodiments, a plurality of different fluorochromes includes 2 or more different fluorochromes, including 3 or more, 4 or more, 5 or more, 6 or more, 7 or more, 8 or more, 9 or more, 10 or more, 11, or more, 12 or more, 13 or more, 14 or more 15 or more, 20 or more, 25 or more and 30 or more different fluorochromes. Fluorescent flow cytometer data may be obtained by any convenient protocol, including those described below.

In some embodiments, methods include generating one or more population clusters based on the determined parameters of analytes (e.g., cells, particles) in the sample. As used herein, a “population”, or “subpopulation” of analytes, such as cells or other particles, generally refers to a group of analytes that possess properties (for example, optical, impedance, or temporal properties) with respect to one or more measured fluorescent parameters such that measured parameter data form a cluster in the data space. Thus, populations are recognized as clusters in the data. Conversely, each data cluster generally is interpreted as corresponding to a population of a particular type of cell or analyte, although clusters that correspond to noise or background typically also are observed. A cluster may be defined in a subset of the dimensions, e.g., with respect to a subset of the measured fluorescent parameters (i.e., fluorochromes), which corresponds to populations that differ in only a subset of the measured parameters or features extracted from the measurements of the sample.

Methods by which flow cytometer data used in the present invention are produced may vary, as desired. For example, a sample having particles (e.g., in a flow stream of a flow cytometer) may be irradiated with light from a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Where methods include irradiating with a broadband light source, broadband light source protocols of interest may include, but are not limited to, a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, an multi-LED integrated white light source, among other broadband light sources or any combination thereof.

In other embodiments, methods include irradiating with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where methods include irradiating with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

Aspects of the present invention include collecting fluorescent light with a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell. In certain embodiments, methods include detecting fluorescence from the sample with one or more fluorescent light detectors, such as 2 or more, such as 3 or more, such as 4 or more, such as 5 or more, such as 6 or more, such as 7 or more, such as 8 or more, such as 9 or more, such as 10 or more, such as 15 or more and including 25 or more fluorescent light detectors. In embodiments, each of the fluorescent light detectors is configured to generate a fluorescence data signal. Fluorescence from the sample may be detected by each fluorescent light detector, independently, over one or more of the wavelength ranges of 200 nm-1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescent light detectors in the subject light detection system. In certain embodiments, methods include detecting wavelengths of light which correspond to the fluorescence peak wavelength of certain fluorochromes present in the sample. In embodiments, fluorescent flow cytometer data is received from one or more fluorescent light detectors (e.g., one or more detection channels), such as 2 or more, such as 3 or more, such as 4 or more, such as 5 or more, such as 6 or more and including 8 or more fluorescent light detectors (e.g., 8 or more detection channels).

In some embodiments, datasets described herein may be subjected to further analysis and or processing. Methods for additional processing that may be employed in conjunction with methods of the present disclosure are described in, e.g., U.S. Pat. Nos. 11,506,593 and 11,674,879; as well as U.S. Patent Application Publication Nos. 2021/0358566 and 2022/0390349, the disclosures of which are incorporated by reference herein in their entirety.

The reference sample(s) and secondary sample(s) may vary. In embodiments, the reference sample(s) and secondary sample(s) are taken from the same organism or type of organism (e.g., species). In some embodiments, the reference sample(s) and secondary sample(s) are produced or prepared in the same manner or using the same protocol. In some instances, the sample analyzed in the instant methods is a biological sample. The term “biological sample” is used in its conventional sense to refer to a whole organism, plant, fungi or a subset of animal tissues, cells or component parts which may in certain instances be found in blood, mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs. Biological samples may be any type of organismic tissue, including both healthy and diseased tissue (e.g., cancerous, malignant, necrotic, etc.). In certain embodiments, the biological sample is a liquid sample, such as blood or derivative thereof, e.g., plasma, tears, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.).

In certain embodiments the source of the sample is a “mammal” or “mammalian”, where these terms are used broadly to describe organisms which are within the class Mammalia, including the orders carnivore (e.g., dogs and cats), Rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

Cells of interest may be targeted for characterized according to a variety of parameters, such as a phenotypic characteristic identified via the attachment of a particular fluorescent label to cells of interest. In some embodiments, the system is configured to deflect analyzed droplets that are determined to include a target cell. A variety of cells may be characterized using the subject methods. Target cells of interest include, but are not limited to, stem cells, T cells, dendritic cells, B Cells, granulocytes, leukemia cells, lymphoma cells, virus cells (e.g., HIV cells), NK cells, macrophages, monocytes, fibroblasts, epithelial cells, endothelial cells, and erythroid cells. Target cells of interest include cells that have a convenient cell surface marker or antigen that may be captured or labelled by a convenient affinity agent or conjugate thereof. For example, the target cell may include a cell surface antigen such as CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71. In some embodiments, the target cell is selected from HIV containing cell, a Treg cell, an antigen-specific T-cell populations, tumor cells or hematopoietic progenitor cells (CD34+) from whole blood, bone marrow or cord blood.

Methods of interest may further include employing particles in research, laboratory testing, or therapy. In some embodiments, the subject methods include obtaining individual cells prepared from a target fluidic or tissue biological sample. For example, the subject methods include obtaining cells from fluidic or tissue samples to be used as a research or diagnostic specimen for diseases such as cancer. Likewise, the subject methods include obtaining cells from fluidic or tissue samples to be used in therapy. A cell therapy protocol is a protocol in which viable cellular material including, e.g., cells and tissues, may be prepared and introduced into a subject as a therapeutic treatment. Conditions that may be treated by the administration of the flow cytometrically sorted sample include, but are not limited to, blood disorders, immune system disorders, organ damage, etc.

A typical cell therapy protocol may include the following steps: sample collection, cell isolation, genetic modification, culture, and expansion in vitro, cell harvesting, sample volume reduction and washing, bio-preservation, storage, and introduction of cells into a subject. The protocol may begin with the collection of viable cells and tissues from source tissues of a subject to produce a sample of cells and/or tissues. The sample may be collected via any suitable procedure that includes, e.g., administering a cell mobilizing agent to a subject, drawing blood from a subject, removing bone marrow from a subject, etc. After collecting the sample, cell enrichment may occur via several methods including, e.g., centrifugation based methods, filter based methods, elutriation, magnetic separation methods, fluorescence-activated cell sorting (FACS), and the like. In some cases, the enriched cells may be genetically modified by any convenient method, e.g., nuclease mediated gene editing. The genetically modified cells can be cultured, activated, and expanded in vitro. In some cases, the cells are preserved, e.g., cryopreserved, and stored for future use where the cells are thawed and then administered to a patient, e.g., the cells may be infused in the patient.

### Systems

Aspects of the invention additionally include systems. Systems of interest include a processor comprising memory operably coupled to the processor, wherein the memory comprises instructions stored thereon, which when executed by the processor, cause the processor to perform methods of the invention (e.g., described above). In particular, the processor is configured to receive a secondary dataset comprising data points collected from a secondary sample, and calculate a second dimensionality reduction for the secondary dataset based on a first dimensionality reduction of a reference dataset comprising data points collected from a reference sample. As noted above, the second dimensionality reduction is compatible with the dimensionally reduced reference dataset.

In some cases, the subject processors are operated in conjunction with programmable logic that may be implemented in hardware, software, firmware, or any combination thereof in order to, e.g., calculate a second dimensionality reduction for the secondary dataset based on a first dimensionality reduction of a reference dataset comprising data points collected from a reference sample. For example, where programmable logic is implemented in software, methods may be realized at least in part by a computer-readable data storage medium comprising program code including instructions that, when executed, is configured to calculate a second dimensionality reduction for the secondary dataset based on a first dimensionality reduction of a reference dataset comprising data points collected from a reference sample. For example, the program code may include instructions for determining data points collected from the reference sample that are closest to each data point collected from the secondary sample with respect to given data parameters, e.g., by calculating k nearest neighbors within the reference dataset for each data point collected from the secondary sample. The processor may additionally be configured to carry out any of the other method steps described above.

The subject programmable logic may be implemented in any of a variety of devices such as specifically programmed event processing computers, wireless communication devices, integrated circuit devices, or the like. In some embodiments, the programable logic may be executed by a specifically programmed processor, which may include one or more processors, such as one or more digital signal processors (DSPs), configurable microprocessors, application specific integrated circuits (ASICs), field programmable gate arrays (FPGAs), or other equivalent integrated or discrete logic circuitry. A combination of computing devices, e.g., a combination of a DSP and a microprocessor, a plurality of microprocessors, one or more microprocessors in conjunction with a DSP core, or any other such configuration in at least partial data connectivity may implement one or more of the features described herein. In certain cases, systems further include a display configured to output the results of the subject methods (e.g., dimensionally reduced plots of data, etc.). The subject display may include, but is not limited to, a monitor, a tablet computer, a smartphone, or other electronic device configured to present graphical interfaces.

In embodiments, the system is configured to analyze the data within a software or an analysis tool for analyzing flow cytometer data, such as FlowJo™ (Ashland, OR). FlowJo™ is a software package developed by FlowJo LLC (a subsidiary of Becton Dickinson) for analyzing flow cytometer data. The software is configured to manage flow cytometer data and produce graphical reports thereon (https://www(dot)flowjo(dot)com/learn/flowjo-university/flowjo). The initial data can be analyzed within the data analysis software or tool (e.g., FlowJo™) by appropriate means, such as manual gating, cluster analysis, or other computational techniques. The instant systems, or a portion thereof, can be implemented as software components of a software for analyzing data, such as FlowJo™. In these embodiments, computer-controlled systems according to the instant disclosure may function as a software “plugin” for an existing software package, such as FlowJo™.

In some embodiments, the subject processor is employed as a part of, or in conjunction with, a flow cytometer. Flow cytometers of interest include a flow cell. The term “flow cell” is described in its conventional sense to refer to a component, such as a cuvette, containing a flow channel having a liquid flow stream for transporting particles in a sheath fluid. Cuvettes of interest include containers having a passage running therethrough. The flow stream may include a liquid sample injected from a sample tube. Flow cells of interest include a light-accessible flow channel. In some instances, the flow cell includes transparent material (e.g., quartz) that permits the passage of light therethrough. In some embodiments, the flow cell is a stream-in-air flow cell in which light interrogation of the particles occurs in free space. In some cases, the flow stream is configured for irradiation with light from one or more light sources at interrogation points. As discussed herein, an “interrogation point” refers to a region within the flow stream in which a particle is irradiated by light from a light source, e.g., for analysis. The size of the interrogation point may vary as desired. For example, where 0 μm represents the axis of light emitted by the light relevant light source, the interrogation zone may range from −100 μm to 100 μm, such as −50 μm to 50 μm, such as −25 μm to 40 μm, and including −15 μm to 30 μm. The flow stream for which the flow channel is configured may include a liquid sample injected from a sample tube. In certain embodiments, the flow stream may include a narrow, rapidly flowing stream of liquid that is arranged such that linearly segregated particles transported therein are separated from each other in a single-file manner. After particles are irradiated in the flow cell, particle-modulated light may be observed.

Any convenient flow cell which propagates a fluidic sample to a sample interrogation region may be employed, where in some embodiments, the flow cell includes is a cylindrical flow cell, a frustoconical flow cell or a flow cell that includes a proximal cylindrical portion defining a longitudinal axis and a distal frustoconical portion which terminates in a flat surface having the orifice that is transverse to the longitudinal axis.

Aspects of the subject systems also includes a light source for irradiating the flow stream at an interrogation point. Any convenient light source may be employed, such as a laser. In embodiments, the laser may be any convenient laser, such as a continuous wave laser. For example, the laser may be a diode laser, such as an ultraviolet diode laser, a visible diode laser and a near-infrared diode laser. In other embodiments, the laser may be a helium-neon (HeNe) laser. In some instances, the laser is a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCl) excimer laser or xenon-fluorine (XeF) excimer laser or a combination thereof. In other instances, the subject flow cytometers include a dye laser, such as a stilbene, coumarin or rhodamine laser. In yet other instances, lasers of interest include a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, the subject flow cytometers include a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

Laser light sources according to certain embodiments may also include one or more optical adjustment components. In certain embodiments, the optical adjustment component is located between the light source and the flow cell, and may include any device that is capable of changing the spatial width of irradiation or some other characteristic of irradiation from the light source, such as for example, irradiation direction, wavelength, beam width, beam intensity and focal spot. Optical adjustment protocols may include any convenient device which adjusts one or more characteristics of the light source, including but not limited to lenses, mirrors, filters, fiber optics, wavelength separators, pinholes, slits, collimating protocols and combinations thereof. In certain embodiments, flow cytometers of interest include one or more focusing lenses. The focusing lens, in one example, may be a de-magnifying lens. In still other embodiments, flow cytometers of interest include fiber optics.

Where the optical adjustment component is configured to move, the optical adjustment component may be configured to be moved continuously or in discrete intervals, such as for example in 0.01 μm or greater increments, such as 0.05 μm or greater, such as 0.1 μm or greater, such as 0.5 μm or greater such as 1 μm or greater, such as 10 μm or greater, such as 100 μm or greater, such as 500 μm or greater, such as 1 mm or greater, such as 5 mm or greater, such as 10 mm or greater and including 25 mm or greater increments.

Any displacement protocol may be employed to move the optical adjustment component structures, such as coupled to a moveable support stage or directly with a motor actuated translation stage, leadscrew translation assembly, geared translation device, such as those employing a stepper motor, servo motor, brushless electric motor, brushed DC motor, micro-step drive motor, high resolution stepper motor, among other types of motors.

The light source may be positioned any suitable distance from the flow cell, such as where the light source and the flow cell are separated by 0.005 mm or more, such as 0.01 mm or more, such as 0.05 mm or more, such as 0.1 mm or more, such as 0.5 mm or more, such as 1 mm or more, such as 5 mm or more, such as 10 mm or more, such as 25 mm or more and including at a distance of 100 mm or more. In addition, the light source may be positioned at any suitable angle relative to the flow cell, such as at an angle ranging from 10 degrees to 90 degrees, such as from 15 degrees to 85 degrees, such as from 20 degrees to 80 degrees, such as from 25 degrees to 75 degrees and including from 30 degrees to 60 degrees, for example at a 90 degree angle.

Systems according to certain embodiments include a plurality of light sources. In some cases, the plurality of light sources includes a plurality of lasers. such as 2 lasers or more, such as 3 lasers or more, such as 4 lasers or more, such as 5 lasers or more, such as 10 lasers or more, and including 15 lasers or more configured to provide laser light for discrete irradiation of the flow stream. Depending on the desired wavelengths of light for irradiating the flow stream, each laser may have a specific wavelength that varies from 200 nm to 1500 nm, such as from 250 nm to 1250 nm, such as from 300 nm to 1000 nm, such as from 350 nm to 900 nm and including from 400 nm to 800 nm. In certain embodiments, lasers of interest may include one or more of a 405 nm laser, a 488 nm laser, a 561 nm laser and a 635 nm laser.

Systems of interest may include one or more detectors for detecting particle-modulated light intensity data. In some embodiments, the particle-modulated light detector(s) include one or more forward-scattered light detectors configured to detect forward-scattered light. For example, the subject particle analyzers may include 1 forward-scattered light detector or multiple forward-scattered light detectors, such as 2 or more, such as 3 or more, such as 4 or more, and including 5 or more. In certain embodiments, particle analyzers include 1 forward-scattered light detector. In other embodiments, particle analyzers include 2 forward-scattered light detectors.

Any convenient detector for detecting collected light may be used in the forward-scattered light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from 0.1 cm² to 8 cm², such as from 0.5 cm² to 7 cm² and including from 1 cm² to 5 cm².

In embodiments, the forward-scattered light detector is configured to measure light continuously or in discrete intervals. In some instances, detectors of interest are configured to take measurements of the collected light continuously. In other instances, detectors of interest are configured to take measurements in discrete intervals, such as measuring light every 0.001 millisecond, every 0.01 millisecond, every 0.1 millisecond, every 1 millisecond, every 10 milliseconds, every 100 milliseconds and including every 1000 milliseconds, or some other interval.

In some instances, systems include one or more side-scattered light detectors for detecting side-scatter wavelengths of light (i.e., light refracted and reflected from the surfaces and internal structures of the particle). In some embodiments, particle analyzers include a single side-scattered light detector. In other embodiments, particle analyzers include multiple side-scattered light detectors, such as 2 or more, such as 3 or more, such as 4 or more, and including 5 or more.

Any convenient detector for detecting collected light may be used in the side-scattered light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from 0.1 cm² to 8 cm², such as from 0.5 cm² to 7 cm² and including from 1 cm² to 5 cm².

In embodiments, the subject systems also include a fluorescent light detector configured to detect one or more fluorescent wavelengths of light. In other embodiments, particle analyzers include multiple fluorescent light detectors such as 2 or more, such as 3 or more, such as 4 or more, 5 or more, 10 or more, 15 or more, and including 20 or more.

Any convenient detector for detecting collected light may be used in the fluorescent light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject particle analyzers include multiple fluorescent light detectors, each fluorescent light detector may be the same, or the collection of fluorescent light detectors may be a combination of different types of detectors. For example, where the subject particle analyzers include two fluorescent light detectors, in some embodiments the first fluorescent light detector is a CCD-type device and the second fluorescent light detector (or imaging sensor) is a CMOS-type device. In other embodiments, both the first and second fluorescent light detectors are CCD-type devices. In yet other embodiments, both the first and second fluorescent light detectors are CMOS-type devices. In still other embodiments, the first fluorescent light detector is a CCD-type device and the second fluorescent light detector is a photomultiplier tube (PMT). In still other embodiments, the first fluorescent light detector is a CMOS-type device and the second fluorescent light detector is a photomultiplier tube. In yet other embodiments, both the first and second fluorescent light detectors are photomultiplier tubes.

In embodiments of the present disclosure, fluorescent light detectors of interest are configured to measure collected light at one or more wavelengths, such as at 2 or more wavelengths, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring light emitted by a sample in the flow stream at 400 or more different wavelengths. In some embodiments, 2 or more detectors in the particle analyzers as described herein are configured to measure the same or overlapping wavelengths of collected light.

In some embodiments, fluorescent light detectors of interest are configured to measure collected light over a range of wavelengths (e.g., 200 nm-1000 nm). In certain embodiments, detectors of interest are configured to collect spectra of light over a range of wavelengths. For example, particle analyzers may include one or more detectors configured to collect spectra of light over one or more of the wavelength ranges of 200 nm-1000 nm. In yet other embodiments, detectors of interest are configured to measure light emitted by a sample in the flow stream at one or more specific wavelengths. For example, particle analyzers may include one or more detectors configured to measure light at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof. In certain embodiments, one or more detectors may be configured to be paired with specific fluorophores, such as those used with the sample in a fluorescence assay.

In some embodiments, one or more of the particle-modulated light detectors includes one or more detector arrays, such as an array of photodiodes. In these embodiments, each detector array may include 4 or more detectors, such as 10 or more detectors, such as 25 or more detectors, such as 50 or more detectors, such as 100 or more detectors, such as 250 or more detectors, such as 500 or more detectors, such as 750 or more detectors and including 1000 or more detectors. For example, the detector may be a photodiode array having 4 or more photodiodes, such as 10 or more photodiodes, such as 25 or more photodiodes, such as 50 or more photodiodes, such as 100 or more photodiodes, such as 250 or more photodiodes, such as 500 or more photodiodes, such as 750 or more photodiodes and including 1000 or more photodiodes.

The detectors may be arranged in any geometric configuration as desired, where arrangements of interest include, but are not limited to a square configuration, rectangular configuration, trapezoidal configuration, triangular configuration, hexagonal configuration, heptagonal configuration, octagonal configuration, nonagonal configuration, decagonal configuration, dodecagonal configuration, circular configuration, oval configuration as well as irregular patterned configurations. The detectors in the detector array may be oriented with respect to the other (as referenced in an X-Z plane) at an angle ranging from 10° to 180°, such as from 15° to 170°, such as from 20° to 160°, such as from 25° to 150°, such as from 30° to 120° and including from 45° to 90°. The detector array may be any suitable shape and may be a rectilinear shape, e.g., squares, rectangles, trapezoids, triangles, hexagons, etc., curvilinear shapes, e.g., circles, ovals, as well as irregular shapes, e.g., a parabolic bottom portion coupled to a planar top portion. In certain embodiments, the detector array has a rectangular-shaped active surface.

In some embodiments, particle analyzers include one or more wavelength separators positioned between the flow cell and the particle-modulated light detector(s). The term “wavelength separator” is used herein in its conventional sense to refer to an optical component that is configured to separate light collected from the sample into predetermined spectral ranges. In some embodiments, particle analyzers include a single wavelength separator. In other embodiments, particle analyzers include a plurality of wavelength separators, such as 2 or more wavelength separators, such as 3 or more, such as 4 or more, such as 5 or more, such as 6 or more, such as 7 or more, such as 8 or more, such as 9 or more, such as 10 or more, such as 15 or more, such as 25 or more, such as 50 or more, such as 75 or more and including 100 or more wavelength separators. In some embodiments, the wavelength separator is configured to separate light collected from the sample into predetermined spectral ranges by passing light having a predetermined spectral range and reflecting one or more remaining spectral ranges of light. In other embodiments, the wavelength separator is configured to separate light collected from the sample into predetermined spectral ranges by passing light having a predetermined spectral range and absorbing one or more remaining spectral ranges of light. In yet other embodiments, the wavelength separator is configured to spatially diffract light collected from the sample into predetermined spectral ranges. Each wavelength separator may be any convenient light separation protocol, such as one or more dichroic mirrors, bandpass filters, diffraction gratings, beam splitters or prisms. In some embodiments, the wavelength separator is a prism. In other embodiments, the wavelength separator is a diffraction grating. In certain embodiments, wavelength separators in the subject light detection systems are dichroic mirrors.

In certain cases, one or more detectors in the system may be considered a trigger sensor (i.e., a sensor that observes the presence of the particle and produces a trigger signal). In some embodiments, the trigger sensor is a forward-scattered light detector (e.g., such as those described above). In other cases, the trigger sensor is an axial light loss (ALL) channel sensor. In such cases, the processor may be configured to calculate a trigger window based on the trigger signal, wherein the trigger window provides a time period during which the particle is expected to pass through a detection zone of the detector, and obtain the baseline noise level at time periods that are outside of the trigger window.

Suitable flow cytometry systems may include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49 (pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ flow cytometer, BD Biosciences FACSCanto™ II flow cytometer, BD Accuri™ flow cytometer, BD Accuri™ C6 Plus flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer, BD Biosciences LSRFortessa™ flow cytometer, BD Biosciences LSRFortessa™ X-20 flow cytometer, BD Biosciences FACSPresto™ flow cytometer, BD Biosciences FACSVia™ flow cytometer and BD Biosciences FACSCalibur™ cell sorter, a BD Biosciences FACSCount™ cell sorter, BD Biosciences FACSLyric™ cell sorter, BD Biosciences Via™ cell sorter, BD Biosciences Influx™ cell sorter, BD Biosciences Jazz™ cell sorter, BD Biosciences Aria™ cell sorter, BD Biosciences FACSAria™ II cell sorter, BD Biosciences FACSAria™ III cell sorter, BD Biosciences FACSAria™ Fusion cell sorter and BD Biosciences FACSMelody™ cell sorter, BD Biosciences FACSymphony™ S6 cell sorter or the like.

In some embodiments, the subject systems are flow cytometric systems, such those described in U.S. Pat. Nos. 10,663,476; 10,620,111; 10,613,017; 10,605,713; 10,585,031; 10,578,542; 10,578,469; 10,481,074; 10,302,545; 10,145,793; 10,113,967; 10,006,852; 9,952,076; 9,933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; 4,987,086; 4,498,766; the disclosures of which are herein incorporated by reference in their entirety. In some cases, flow cytometry systems of the invention are clustered wavelength division (CWD) systems. CWD systems are described in, for example, U.S. Patent Application Publication No. 2021/0247293; the disclosure of which is herein incorporated by reference in its entirety.

In certain instances, flow cytometry systems of the invention are configured for imaging particles in a flow stream by fluorescence imaging using radiofrequency tagged emission (FIRE), such as those described in Diebold, et al. Nature Photonics Vol. 7(10); 806-810 (2013) as well as described in U.S. Pat. Nos. 9,423,353; 9,784,661; 9,983,132; 10,006,852; 10,078,045; 10,036,699; 10,222,316; 10,288,546; 10,324,019; 10,408,758; 10,451,538; 10,620,111; and U.S. Patent Publication Nos. 2017/0133857; 2017/0328826; 2017/0350803; 2018/0275042; 2019/0376895 and 2019/0376894 the disclosures of which are herein incorporated by reference.

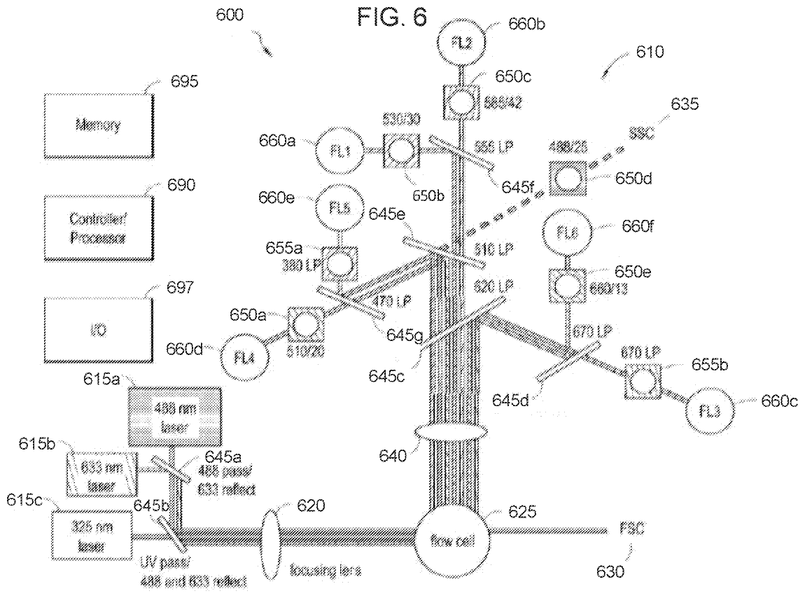

FIG. 6 shows a system 600 for flow cytometry in accordance with an illustrative embodiment of the present invention. The system 600 includes a flow cytometer 610, a controller/processor 690 and a memory 695. The flow cytometer 610 includes one or more excitation lasers 615a-615c, a focusing lens 620, a flow chamber 625, a forward-scatter detector 630, a side-scatter detector 635, a fluorescence collection lens 640, one or more beam splitters 645a-645g, one or more bandpass filters 650a-650e, one or more longpass (“LP”) filters 655a-655b, and one or more fluorescent detectors 660a-660f.

The excitation lasers 615a-c emit light in the form of a laser beam. The wavelengths of the laser beams emitted from excitation lasers 615a-615c are 488 nm, 633 nm, and 325 nm, respectively, in the example system of FIG. 6. The laser beams are first directed through one or more of beam splitters 645a and 645b. Beam splitter 645a transmits light at 488 nm and reflects light at 633 nm. Beam splitter 645b transmits UV light (light with a wavelength in the range of 10 to 400 nm) and reflects light at 488 nm and 633 nm.

The laser beams are then directed to a focusing lens 620, which focuses the beams onto the portion of a fluid stream where particles of a sample are located, within the flow chamber 625. The flow chamber is part of a fluidics system which directs particles, typically one at a time, in a stream to the focused laser beam for interrogation. The flow chamber can comprise a flow cell in a benchtop cytometer or a nozzle tip in a stream-in-air cytometer.

The light from the laser beam(s) interacts with the particles in the sample by diffraction, refraction, reflection, scattering, and absorption with re-emission at various different wavelengths depending on the characteristics of the particle such as its size, internal structure, and the presence of one or more fluorescent molecules attached to or naturally present on or in the particle. The fluorescence emissions as well as the diffracted light, refracted light, reflected light, and scattered light may be routed to one or more of the forward-scatter detector 630, the side-scatter detector 635, and the one or more fluorescent detectors 660a-660f through one or more of the beam splitters 645c-645g, the bandpass filters 650a-650e, the longpass filters 655a-655b, and the fluorescence collection lens 640.

The fluorescence collection lens 640 collects light emitted from the particle-laser beam interaction and routes that light towards one or more beam splitters and filters. Bandpass filters, such as bandpass filters 650a-650e, allow a narrow range of wavelengths to pass through the filter. For example, bandpass filter 650a is a 510/20 filter. The first number represents the center of a spectral band. The second number provides a range of the spectral band. Thus, a 510/20 filter extends 10 nm on each side of the center of the spectral band, or from 500 nm to 520 nm. Shortpass filters transmit wavelengths of light equal to or shorter than a specified wavelength. Longpass filters, such as longpass filters 655a-655b, transmit wavelengths of light equal to or longer than a specified wavelength of light. For example, longpass filter 655b, which is a 670 nm longpass filter, transmits light equal to or longer than 670 nm. Filters are often selected to optimize the specificity of a detector for a particular fluorescent dye. The filters can be configured so that the spectral band of light transmitted to the detector is close to the emission peak of a fluorescent dye.

The forward-scatter detector 630 is positioned slightly off axis from the direct beam through the flow cell and is configured to detect diffracted light, the excitation light that travels through or around the particle in mostly a forward direction. The intensity of the light detected by the forward-scatter detector is dependent on the overall size of the particle. The forward-scatter detector can include a photodiode. The side-scatter detector 635 is configured to detect refracted and reflected light from the surfaces and internal structures of the particle that tends to increase with increasing particle complexity of structure. The fluorescence emissions from fluorescent molecules associated with the particle can be detected by the one or more fluorescent detectors 660a-660f. The side-scatter detector 635 and fluorescent detectors can include photomultiplier tubes. The signals detected at the forward-scatter detector 630, the side-scatter detector 635 and the fluorescent detectors can be converted to electronic signals (voltages) by the detectors. This data can provide information about the sample.

One of skill in the art will recognize that a flow cytometer in accordance with an embodiment of the present invention is not limited to the flow cytometer depicted in FIG. 6, but can include any flow cytometer known in the art. For example, a flow cytometer may have any number of lasers, beam splitters, filters, and detectors at various wavelengths and in various different configurations.

In operation, cytometer operation is controlled by a controller/processor 690, and the measurement data from the detectors can be stored in the memory 695 and processed by the controller/processor 690. Although not shown explicitly, the controller/processor 690 is coupled to the detectors to receive the output signals therefrom, and may also be coupled to electrical and electromechanical components of the flow cytometer 610 to control the lasers, fluid flow parameters, and the like. Input/output (I/O) capabilities 697 may be provided also in the system. The memory 695, controller/processor 690, and 1/O 697 may be entirely provided as an integral part of the flow cytometer 610. In such an embodiment, a display may also form part of the I/O capabilities 697 for presenting experimental data to users of the cytometer 610. Alternatively, some or all of the memory 695 and controller/processor 690 and 1/O capabilities may be part of one or more external devices such as a general purpose computer. In some embodiments, some or all of the memory 695 and controller/processor 690 can be in wireless or wired communication with the cytometer 610. The controller/processor 690 in conjunction with the memory 695 and the I/O 697 can be configured to perform various functions related to the preparation and analysis of a flow cytometer experiment.

The system illustrated in FIG. 6 includes six different detectors that detect fluorescent light in six different wavelength bands (which may be referred to herein as a “filter window” for a given detector) as defined by the configuration of filters and/or splitters in the beam path from the flow cell 625 to each detector. Different fluorescent molecules used for a flow cytometer experiment will emit light in their own characteristic wavelength bands. The particular fluorescent labels used for an experiment and their associated fluorescent emission bands may be selected to generally coincide with the filter windows of the detectors. The I/O 697 can be configured to receive data regarding a flow cytometer experiment having a panel of fluorescent labels and a plurality of cell populations having a plurality of markers, each cell population having a subset of the plurality of markers. The I/O 697 can also be configured to receive biological data assigning one or more markers to one or more cell populations, marker density data, emission spectrum data, data assigning labels to one or more markers, and cytometer configuration data. Flow cytometer experiment data, such as label spectral characteristics and flow cytometer configuration data can also be stored in the memory 695. The controller/processor 690 can be configured to evaluate one or more assignments of labels to markers.

In some embodiments, the subject systems are particle sorting systems that are configured to sort particles with an enclosed particle sorting module, such as those described in U.S. Patent Publication No. 2017/0299493, filed on Mar. 28, 2017, the disclosure of which is incorporated herein by reference. In certain embodiments, particles (e.g., cells) of the sample are sorted using a sort decision module having a plurality of sort decision units, such as those described in U.S. Patent Publication No. 2020/0256781, filed on Dec. 23, 2019, the disclosure of which is incorporated herein by reference. In some embodiments, systems for sorting components of a sample include a particle sorting module having deflection plates, such as described in U.S. Patent Publication No. 2017/0299493, filed on Mar. 28, 2017, the disclosure of which is incorporated herein by reference.

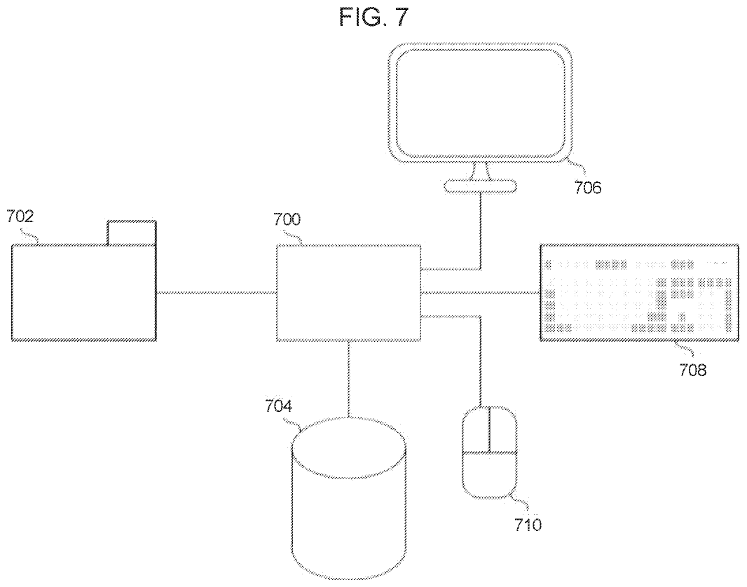

FIG. 7 shows a functional block diagram for one example of a system, having a processor 700, for analyzing and displaying biological events. A processor 700 can be configured to implement a variety of processes for controlling graphic display of biological events. A flow cytometer or sorting system 702 can be configured to acquire biological event data. For example, a flow cytometer can generate flow cytometric event data (e.g., particle-modulated light data). The flow cytometer 702 can be configured to provide biological event data to the processor 700. A data communication channel can be included between the flow cytometer 702 and the processor 700. The biological event data can be provided to the processor 700 via the data communication channel.

The processor 700 can be configured to receive biological event data from the flow cytometer 702. The biological event data received from the flow cytometer 702 can include flow cytometric event data. The processor 700 is configured to evaluate the data received from the flow cytometer 702, e.g., as discussed above. Processor 700 may be configured to perform the subject methods, e.g., receive a secondary dataset comprising data points collected from a secondary sample (optionally by flow cytometer or sorting system 702), and calculate a second dimensionality reduction for the secondary dataset based on a first dimensionality reduction of a reference dataset comprising data points collected from a reference sample.

The processor 700 can be configured to provide a graphical display including a biological event data to a display device 706. For example, processor 700 may provide the ideal gains calculated for each of the detectors (e.g., fluorescent light detectors 360a-e) in the system to the graphical display device 706. These ideal gains may be outputted in the form of, e.g., tube target values (TTVs). The processor 700 can be further configured to render a region of interest as a gate around a population of biological event data shown by the display device 706, overlaid upon the first plot, for example. In some embodiments, the gate can be a logical combination of one or more graphical regions of interest drawn upon a single parameter histogram or bivariate plot. In some embodiments, the display can be used to display particle parameters or saturated detector data.

The processor 700 can be further configured to display the biological event data on the display device 706 within the gate differently from other events in the biological event data outside of the gate. For example, the processor 700 can be configured to render the color of biological event data contained within the gate to be distinct from the color of biological event data outside of the gate. The display device 706 can be implemented as a monitor, a tablet computer, a smartphone, or other electronic device configured to present graphical interfaces.

The processor 700 can be configured to receive a gate selection signal identifying the gate from a first input device. For example, the first input device can be implemented as a mouse 710. The mouse 710 can initiate a gate selection signal to the processor 700 identifying the gate to be displayed on or manipulated via the display device 706 (e.g., by clicking on or in the desired gate when the cursor is positioned there). In some implementations, the first device can be implemented as the keyboard 708 or other means for providing an input signal to the processor 700 such as a touchscreen, a stylus, an optical detector, or a voice recognition system. Some input devices can include multiple inputting functions. In such implementations, the inputting functions can each be considered an input device. For example, as shown in FIG. 7, the mouse 710 can include a right mouse button and a left mouse button, each of which can generate a triggering event.

The triggering event can cause the processor 700 to alter the manner in which the data is displayed, which portions of the data is actually displayed on the display device 706, and/or provide input to further processing such as selection of a population of interest for particle sorting.

In some embodiments, the processor 700 can be configured to detect when gate selection is initiated by the mouse 710. The processor 700 can be further configured to automatically modify plot visualization to facilitate the gating process. The modification can be based on the specific distribution of biological event data received by the processor 700. In some embodiments, the processor 700 expands the first gate such that a second gate is generated (e.g., as discussed above).

The processor 700 can be connected to a storage device 704. The storage device 704 can be configured to receive and store biological event data from the processor 700. The storage device 704 can also be configured to receive and store flow cytometric event data from the processor 700. The storage device 704 can be further configured to allow retrieval of biological event data, such as flow cytometric event data, by the processor 700.

The display device 706 can be configured to receive display data from the processor 700. The display data can comprise plots of biological event data and gates outlining sections of the plots. The display device 706 can be further configured to alter the information presented according to input received from the processor 700 in conjunction with input from the flow cytometer 702, the storage device 704, the keyboard 708, and/or the mouse 710.

In some implementations the processor 700 can generate a user interface to receive example events for sorting. For example, the user interface can include a mechanism for receiving example events or example images. The example events or images or an example gate can be provided prior to collection of event data for a sample or based on an initial set of events for a portion of the sample.

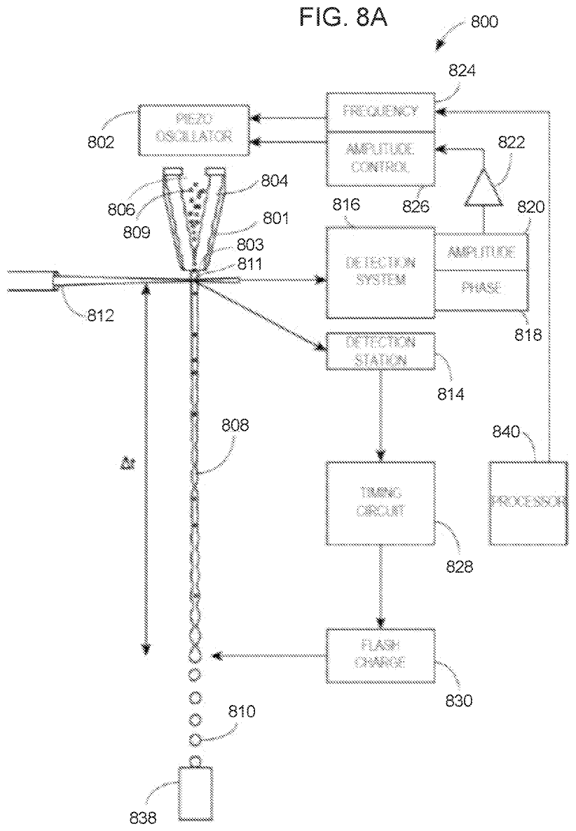

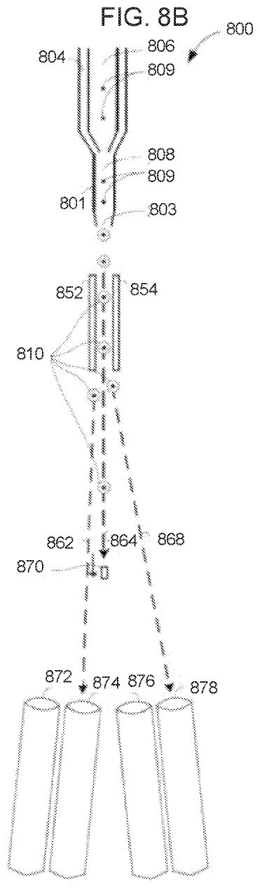

FIG. 8A is a schematic drawing of a particle sorter system 800 (e.g., the flow cytometer 702) in accordance with one embodiment presented herein. In some embodiments, the particle sorter system 800 is a cell sorter system. As shown in FIG. 8A, a drop formation transducer 802 (e.g., piezo-oscillator) is coupled to a fluid conduit 801, which can be coupled to, can include, or can be, a nozzle 803. Within the fluid conduit 801, sheath fluid 804 hydrodynamically focuses a sample fluid 806 comprising particles 809 into a moving fluid column 808 (e.g., a stream). Within the moving fluid column 808, particles 809 (e.g., cells) are lined up in single file to cross a monitored area 811 (e.g., where laser-stream intersect), irradiated by an irradiation source 812 (e.g., a laser). Vibration of the drop formation transducer 802 causes moving fluid column 808 to break into a plurality of drops 810, some of which contain particles 809.

In operation, a detection station 814 (e.g., an event detector) identifies when a particle of interest (or cell of interest) crosses the monitored area 811. Detection station 814 feeds into a timing circuit 828, which in turn feeds into a flash charge circuit 830. At a drop break off point, informed by a timed drop delay (Δt), a flash charge can be applied to the moving fluid column 808 such that a drop of interest carries a charge. The drop of interest can include one or more particles or cells to be sorted. The charged drop can then be sorted by activating deflection plates (not shown) to deflect the drop into a vessel such as a collection tube or a multi-well or microwell sample plate where a well or microwell can be associated with drops of particular interest. As shown in FIG. 8A, the drops can be collected in a drain receptacle 838.

A detection system 816 (e.g., a drop boundary detector) serves to automatically determine the phase of a drop drive signal when a particle of interest passes the monitored area 811. An exemplary drop boundary detector is described in U.S. Pat. No. 7,679,039, which is incorporated herein by reference in its entirety. The detection system 816 allows the instrument to accurately calculate the place of each detected particle in a drop. The detection system 816 can feed into an amplitude signal 820 and/or phase 818 signal, which in turn feeds (via amplifier 822) into an amplitude control circuit 826 and/or frequency control circuit 824. The amplitude control circuit 826 and/or frequency control circuit 824, in turn, controls the drop formation transducer 802. The amplitude control circuit 826 and/or frequency control circuit 824 can be included in a control system.

In some implementations, sort electronics (e.g., the detection system 816, the detection station 814 and a processor 840) can be coupled with a memory configured to store the detected events and a sort decision based thereon. The sort decision can be included in the event data for a particle. In some implementations, the detection system 816 and the detection station 814 can be implemented as a single detection unit or communicatively coupled such that an event measurement can be collected by one of the detection system 816 or the detection station 814 and provided to the non-collecting element.

FIG. 8B is a schematic drawing of a particle sorter system, in accordance with one embodiment presented herein. The particle sorter system 800 shown in FIG. 8B, includes deflection plates 852 and 854. A charge can be applied via a stream-charging wire in a barb. This creates a stream of droplets 810 containing particles 809 for analysis. The particles can be illuminated with one or more light sources (e.g., lasers) to generate light scatter and fluorescence information. The information for a particle is analyzed such as by sorting electronics or other detection system (not shown in FIG. 8B). The deflection plates 852 and 854 can be independently controlled to attract or repel the charged droplet to guide the droplet toward a destination collection vessel (e.g., one of 872, 874, 876, or 878). As shown in FIG. 8B, the deflection plates 852 and 854 can be controlled to direct a particle along a first path 862 toward the vessel 874 or along a second path 868 toward the vessel 878. If the particle is not of interest (e.g., does not exhibit scatter or illumination information within a specified sort range), deflection plates may allow the particle to continue along a flow path 864. Such uncharged droplets may pass into a waste receptacle such as via aspirator 870.

The sorting electronics can be included to initiate collection of measurements, receive fluorescence signals for particles, and determine how to adjust the deflection plates to cause sorting of the particles. Example implementations of the embodiment shown in FIG. 8B include the BD FACSAria™ line of flow cytometers commercially provided by Becton, Dickinson and Company (Franklin Lakes, NJ).

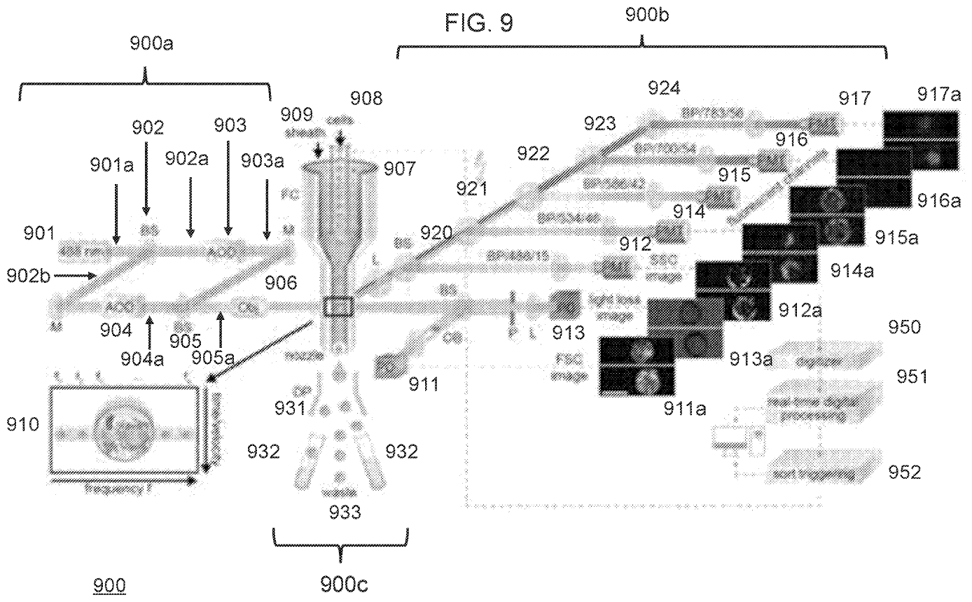

In certain embodiments, systems are a fluorescence imaging using radiofrequency tagged emission image-enabled particle sorter, such as depicted in FIG. 9. Particle sorter 900 includes a light irradiation component 900a which includes light source 901 (e.g., 488 nm laser) which generates output beam of light 901a that is split with beamsplitter 902 into beams 902a and 902b. Light beam 902a is propagated through acousto-optic device (e.g., an acousto-optic deflector, AOD) 903 to generate an output beam 903a having one or more angularly deflected beams of light. In some instances, output beam 903a generated from acousto-optic device 903 includes a local oscillator beam and a plurality of radiofrequency comb beams. Light beam 902b is propagated through acousto-optic device (e.g., an acousto-optic deflector, AOD) 904 to generate an output beam 904a having one or more angularly deflected beams of light. In some instances, output beam 904a generated from acousto-optic device 904 includes a local oscillator beam and a plurality of radiofrequency comb beams. Output beams 903a and 904a generated from acousto-optic devices 903 and 904, respectively are combined with beamsplitter 905 to generate output beam 905a which is conveyed through an optical component 906 (e.g., an objective lens) to irradiate particles in flow cell 907. In certain embodiments, acousto-optic device 903 (AOD) splits a single laser beam into an array of beamlets, each having different optical frequency and angle. Second AOD 904 tunes the optical frequency of a reference beam, which is then overlapped with the array of beamlets at beam combiner 905. In certain embodiments, the light irradiation system having a light source and acousto-optic device can also include those described in Schraivogel, et al. (“High-speed fluorescence image-enabled cell sorting” Science (2022), 375 (6578): 315-320) and United States Patent Publication No. 2021/0404943, the disclosure of which is herein incorporated by reference.

Output beam 905a irradiates sample particles 908 propagating through flow cell 907 (e.g., with sheath fluid 909) at irradiation region 910. As shown in irradiation region 910, a plurality of beams (e.g., angularly deflected radiofrequency shifted beams of light depicted as dots across irradiation region 910) overlaps with a reference local oscillator beam (depicted as the shaded line across irradiation region 910). Due to their differing optical frequencies, the overlapping beams exhibit a beating behavior, which causes each beamlet to carry a sinusoidal modulation at a distinct frequency f₁₋ₙ.

Light from the irradiated sample is conveyed to light detection system 900b that includes a plurality of photodetectors. Light detection system 900b includes forward scattered light photodetector 911 for generating forward scatter images 911a and a side scattered light photodetector 912 for generating side scatter images 912a. Light detection system 900b also includes brightfield photodetector 913 for generating light loss images 913a. In some embodiments, forward scatter detector 911 and side scatter detector 912 are photodiodes (e.g., avalanche photodiodes, APDs). In some instances, brightfield photodetector 913 is a photomultiplier tube (PMT). Fluorescence from the irradiated sample is also detected with fluorescence photodetectors 914-917. In some instances, photodetectors 914-917 are photomultiplier tubes. Light from the irradiated sample is directed to the side scatter detection channel 912 and fluorescence detection channels 914-917 through beamsplitter 920. Light detection system 900b includes bandpass optical components 921, 922, 923 and 924 (e.g., dichroic mirrors) for propagating predetermined wavelength of light to photodetectors 914-917. In some instances, optical component 921 is a 534 nm/40 nm bandpass. In some instances, optical component 922 is a 586 nm/42 nm bandpass. In some instances, optical component 923 is a 700 nm/54 nm bandpass. In some instances, optical component 924 is a 783 nm/56 nm bandpass. The first number represents the center of a spectral band. The second number provides a range of the spectral band. Thus, a 510/20 filter extends 10 nm on each side of the center of the spectral band, or from 500 nm to 520 nm.

Data signals generated in response to light detected in scattered light detection channels 911 and 912, brightfield light detection channel 913 and fluorescence detection channels 914-917 are processed by real-time digital processing with processors 950 and 951. Images 911a-917a can be generated in each light detection channel based on the data signals generated in processors 950 and 951. Image-enabled sorting is performed in response to a sort signal generated in sort trigger 952. Sorting component 900c includes deflection plates 931 for deflecting particles into sample containers 932 or to waste stream 933. In some instances, sort component 900c is configured to sort particles with an enclosed particle sorting module, such as those described in U.S. Patent Publication No. 2017/0299493, filed on Mar. 28, 2017, the disclosure of which is incorporated herein by reference. In certain embodiments, sorting component 900c includes a sort decision module having a plurality of sort decision units, such as those described in U.S. Patent Publication No. 2020/0256781, the disclosure of which is incorporated herein by reference.

### Non-Transitory Computer Readable Storage Media

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, flash drive, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Java, Python, Visual Basic, and C++, as well as many others.

In some embodiments, computer readable storage media of interest include a computer program stored thereon, where the computer program when loaded on the computer includes instructions for practicing methods of the invention described herein, i.e., receiving a secondary dataset comprising data points collected from a secondary sample, and calculating a second dimensionality reduction for the secondary dataset based on a first dimensionality reduction of a reference dataset comprising data points collected from a reference sample. The second dimensionality reduction calculated by the computer readable storage media of interest is compatible with the dimensionally reduced reference dataset.

### Computer-Controlled Systems

Aspects of the present disclosure further include computer-controlled systems, where the systems include one or more computers for complete automation or partial automation. In some embodiments, systems include a computer having a non-transitory computer readable storage medium with a computer program stored thereon, where the computer program when loaded on the computer includes instructions for practicing methods of the invention described herein, i.e., receiving a secondary dataset comprising data points collected from a secondary sample, and calculating a second dimensionality reduction for the secondary dataset based on a first dimensionality reduction of a reference dataset comprising data points collected from a reference sample. The second dimensionality reduction calculated is compatible with the dimensionally reduced reference dataset.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, or a diskette drive. Such types of memory storage devices typically read from, and/or write to, a program storage medium (not shown) such as a compact disk. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, Wi-Fi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, a USB-C port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or Wi-Fi connection to the internet at a Wi-Fi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows® NT®, Windows® XP, Windows® 7, Windows® 8, Windows® 10, iOS®, macOS®, Linux®, Ubuntu®, Fedora®, OS/400®, i5/OS®, IBM i®, Android®, SGI IRIX®, Oracle Solaris® and others.

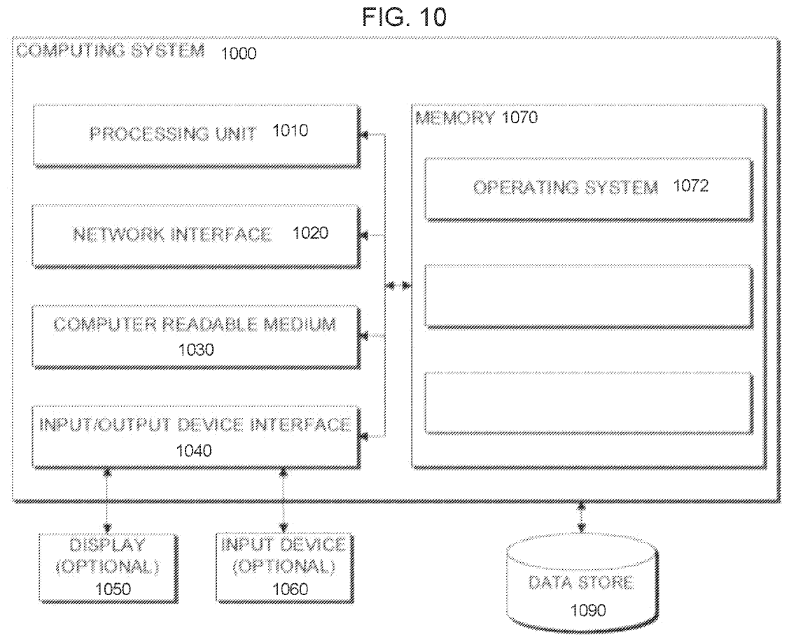

FIG. 10 depicts a general architecture of an example computing device 1000 according to certain embodiments. The general architecture of the computing device 1000 depicted in FIG. 10 includes an arrangement of computer hardware and software components. It is not necessary, however, that all of these generally conventional elements be shown in order to provide an enabling disclosure. As illustrated, the computing device 1000 includes a processing unit 1010, a network interface 1020, a computer readable medium drive 1030, an input/output device interface 1040, a display 1050, and an input device 1060, all of which may communicate with one another by way of a communication bus. The network interface 1020 may provide connectivity to one or more networks or computing systems. The processing unit 1010 may thus receive information and instructions from other computing systems or services via a network. The processing unit 1010 may also communicate to and from memory 1070 and further provide output information for an optional display 1050 via the input/output device interface 1040. For example, an analysis software (e.g., data analysis software or program such as FlowJo™) stored as executable instructions in the non-transitory memory of the analysis system can display the flow cytometry event data to a user. The input/output device interface 1040 may also accept input from the optional input device 1060, such as a keyboard, mouse, digital pen, microphone, touch screen, gesture recognition system, voice recognition system, gamepad, accelerometer, gyroscope, or other input device.

The memory 1070 may contain computer program instructions (grouped as modules or components in some embodiments) that the processing unit 1010 executes in order to implement one or more embodiments. The memory 1070 generally includes RAM, ROM and/or other persistent, auxiliary or non-transitory computer-readable media. The memory 1070 may store an operating system 1072 that provides computer program instructions for use by the processing unit 1010 in the general administration and operation of the computing device 1000. Data may be stored in data storage device 1090. The memory 1070 may further include computer program instructions and other information for implementing aspects of the present disclosure.

### Utility

The present methods, systems and computer readable media may be employed where it is desirable to minimize the need to collect all samples up front and concatenate them in order to calculate a dimensionality reduction. The invention enables a workflow where users collect one or more reference samples, calculate dimensionality reduction on this/these sample(s), and use them as training (reference) to calculate a compatible dimensionality reduction of new samples in the future. The methods, systems, and computer readable media may also be employed where it is desirable to have quality scores designed to indicate whether data is sufficiently stable over time.

Embodiments of the invention find use in applications where cells prepared from a biological sample may be desired for research, laboratory testing or for use in therapy. In some embodiments, the subject methods and devices may facilitate obtaining individual cells prepared from a target fluidic or tissue biological sample. For example, the subject methods and systems facilitate obtaining cells from fluidic or tissue samples to be used as a research or diagnostic specimen for diseases such as cancer. Likewise, the subject methods and systems may facilitate obtaining cells from fluidic or tissue samples to be used in therapy. Methods and devices of the present disclosure allow for separating and collecting cells from a biological sample (e.g., organ, tissue, tissue fragment, fluid) with enhanced efficiency and low cost as compared to traditional flow cytometry systems.

### Kits

Aspects of the present disclosure further include kits, where kits include storage media such as a magneto-optical disk, CD-ROM, CD-R, magnetic tape, non-volatile memory card, ROM, DVD-ROM, Blue-ray disk, solid state disk, and network attached storage (NAS). Any of these program storage media, or others now in use or that may later be developed, may be included in the subject kits. In embodiments, the program storage media include instructions for dimensionality reduction via the methods described herein. In embodiments, the instructions contained on computer readable media provided in the subject kits, or a portion thereof, can be implemented as software components of a software for analyzing data. In these embodiments, computer-controlled systems according to the instant disclosure may function as a software “plugin” for an existing software package (e.g., FlowJo™).

In addition to the above components, the subject kits may further include (in some embodiments) instructions, e.g., for installing the plugin to the existing software package. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

Notwithstanding the appended claims, the invention may also be defined by the following clauses:

1. A computer-implemented method of dimensionality reduction, the method comprising, via a processor:
receiving a secondary dataset comprising data points collected from a secondary sample; and
calculating a second dimensionality reduction for the secondary dataset based on a first dimensionality reduction of a reference dataset comprising data points collected from a reference sample, wherein the second dimensionality reduction is compatible with the dimensionally reduced reference dataset.

2. The computer-implemented method according to Clause 1, wherein calculating the second dimensionality reduction comprises determining data points collected from the reference sample that are closest to each data point collected from the secondary sample with respect to given data parameters.

3. The computer-implemented method according to Clause 2, wherein calculating the second dimensionality reduction comprises calculating k nearest neighbors within the reference dataset for each data point collected from the secondary sample.

4. The computer-implemented method according to Clause 3, wherein k ranges from 1 to m, where m is half of the number of data points in the reference sample.

5. The computer-implemented method according to Clause 4, wherein k ranges from 2 to 5.

6. The computer-implemented method according to any one of Clauses 3 to 5, wherein the method comprises calculating the k nearest neighbors using a vantage-point tree, a k-dimensional tree, ball tree, cover tree, locality-sensitive hashing, hierarchical navigable small world, approximate nearest neighbors with random projection trees, GPU-based KNN search, or a brute force KNN search.

7. The computer-implemented method according to any one of Clauses 3 to 6, further comprising calculating a distance between each data point collected from the secondary sample and each nearest neighbor of the k nearest neighbors.

8. The computer-implemented method according to Clause 7, wherein the distance is a Manhattan distance.

9. The computer-implemented method according to Clause 7, wherein the distance is a Euclidean distance.

10. The computer-implemented method according to Clause 7, wherein the distance is a Chebyshev distance.

11. The computer-implemented method according to Clause 7, wherein the distance is a Minkowski distance.

12. The computer-implemented method according to Clause 7, wherein the distance is a cosine distance.

13. The computer-implemented method according to any one of Clauses 7 to 12, wherein the method comprises calculating the second dimensionality reduction based on a weighted average of positions of the k nearest neighbors.

14. The computer-implemented method according to Clause 13, further comprising calculating the weighted average using a weight obtained for each nearest neighbor of the k nearest neighbors based on the distance.

15. The computer-implemented method according to Clause 14, wherein the method comprises obtaining the weight as follows:

$${w}_{i} = \frac{1 + a}{1 + {d}_{i} + b}$$

wherein:
wᵢ is the weight;
dᵢ is the distance; and
a and b are real numbers.

16. The computer-implemented method according to Clause 14, wherein the method comprises obtaining the weight as follows:

$${w}_{i} = f ( \frac{1 + a}{1 + {d}_{i} + b} )$$

wherein:
f is any monotonic entirely non-decreasing function;
wᵢ is the weight;
dᵢ is the distance; and
a and b are real numbers.

17. The computer-implemented method according to Clause 14, wherein the method comprises obtaining the weight as follows:

$${w}_{i} = \frac{1}{1 + {d}_{i}}$$

wherein:
wᵢ is the weight; and
dᵢ is the distance.

18. The computer-implemented method according to any one of the preceding clauses, wherein the secondary dataset comprises data points associated with a plurality of parameters that are matched to the reference dataset.

19. The computer-implemented method according to Clause 18, further comprising creating derived parameters for the secondary dataset based on parameters of the dimensionally reduced reference dataset.

20. The computer-implemented method according to Clause 19, wherein the derived parameters are fluorescence parameters, scatter parameters, imaging parameters or categorical parameters.

21. The computer-implemented method according to Clause 20, wherein the derived parameters are fluorescence parameters.

22. The computer-implemented method according to any one of Clauses 19 to 21, wherein creating the derived parameters for the secondary dataset comprises linear interpolation.

23. The computer-implemented method according to any one of Clauses 7 to 22, further comprising calculating an input quality score indicating an extent to which each data point collected from the secondary sample is related to the data points within the reference dataset.

24. The computer-implemented method according to Clause 23, wherein calculating the input quality score comprises obtaining a normalized average distance of each data point collected from the secondary sample to the k nearest neighbors.

25. The computer-implemented method according to any one of Clauses 7 to 24, further comprising calculating an output quality score measuring the separation of the k nearest neighbors in the dimensionally reduced first dataset.

26. The computer-implemented method according to Clause 25, wherein calculating the output quality score comprises obtaining a normalized average distance of each of the k nearest neighbors within the dimensionally reduced reference dataset.

27. The computer-implemented method according to any one of the preceding clauses, wherein the reference and secondary datasets are comprised of flow cytometer data.

28. The computer-implemented method according to any one of the preceding clauses, wherein the reference dataset is a uniformly down-sampled reference dataset or a density-based down-sampled reference dataset.

29. The computer-implemented method according to any one of the preceding clauses, wherein the method comprises:
receiving the reference dataset; and
performing the first dimensionality reduction.

30. The computer-implemented method according to any one of the preceding clauses, further comprising transforming the reference dataset and the secondary dataset.

31. The computer-implemented method according to Clause 30, wherein the method comprises transforming parameters in the reference dataset and parameters in the secondary dataset with a linear function, logarithmic function, a hyperbolic arcsine function, or a biexponential function.

32. The computer-implemented method according to any one of the preceding clauses, wherein the method comprises calculating a dimensionality reduction for a plurality of secondary datasets comprising data points collected from a plurality of secondary samples.

33. A system comprising a processor comprising memory operably coupled to the processor, wherein the memory comprises instructions stored thereon, which when executed by the processor, cause the processor to:
receive a secondary dataset comprising data points collected from a secondary sample; and
calculate a second dimensionality reduction for the secondary dataset based on a first dimensionality reduction of a reference dataset comprising data points collected from a reference sample, wherein the second dimensionality reduction is compatible with the dimensionally reduced reference dataset.

34. The system according to Clause 33, wherein calculating the second dimensionality reduction comprises determining data points collected from the reference sample that are closest to each data point collected from the secondary sample with respect to given data parameters.

35. The system according to Clause 34, wherein calculating the second dimensionality reduction comprises calculating k nearest neighbors within the reference dataset for each data point collected from the secondary sample.

36. The system according to Clause 35, wherein k ranges from 1 to m, where m is half of the number of data points in the reference sample.

37. The system according to Clause 36, wherein k ranges from 2 to 5.

38. The system according to any one of Clauses 35 to 37, wherein the processor is configured to calculate the k nearest neighbors using a vantage-point tree, a k-dimensional tree, ball tree, cover tree, locality-sensitive hashing, hierarchical navigable small world, approximate nearest neighbors with random projection trees, GPU-based KNN search, or a brute force KNN search.

39. The system according to any one of Clauses 35 to 38, wherein the processor is configured to calculate a distance between each data point collected from the secondary sample and each nearest neighbor of the k nearest neighbors.

40. The system according to Clause 39, wherein the distance is a Manhattan distance.

41. The system according to Clause 39, wherein the distance is a Euclidean distance.

42. The system according to Clause 39, wherein the distance is a Chebyshev distance.

43. The system according to Clause 39, wherein the distance is a Minkowski distance.

44. The system according to Clause 39, wherein the distance is a cosine distance.

45. The system according to any one of Clauses 39 to 44, wherein the processor is configured to calculate the second dimensionality reduction based on a weighted average of positions of the k nearest neighbors.

46. The system according to Clause 45, wherein the processor is configured to calculate the weighted average using a weight obtained for each nearest neighbor of the k nearest neighbors based on the distance.

47. The system according to Clause 46, wherein the processor is configured to obtain the weight as follows:

$${w}_{i} = \frac{1 + a}{1 + {d}_{i} + b}$$

wherein:
wᵢ is the weight;
dᵢ is the distance; and
a and b are real numbers.

48. The system according to Clause 46, wherein the processor is configured to obtain the weight as follows:

$${w}_{i} = f ( \frac{1 + a}{1 + {d}_{i} + b} )$$

wherein:
f is any monotonic entirely non-decreasing function;
wᵢ is the weight;
dᵢ is the distance; and
a and b are real numbers.

49. The system according to Clause 46, wherein the processor is configured to obtain the weight as follows:

$${w}_{i} = \frac{1}{1 + {d}_{i}}$$

wherein:
wᵢ is the weight; and
dᵢ is the distance.

50. The system according to any one of Clauses 33 to 49, wherein the secondary dataset comprises data points associated with a plurality of parameters that are matched to the reference dataset.

51. The system according to Clause 50, wherein the processor is configured to create derived parameters for the secondary dataset based parameters of the dimensionally reduced reference dataset.

52. The system according to Clause 51, wherein the derived parameters are fluorescence parameters, scatter parameters, imaging parameters or categorical parameters.

53. The system according to Clause 52, wherein the derived parameters are fluorescence parameters.

54. The system according to any one of Clauses 51 to 53, wherein creating the derived parameters for the secondary dataset comprises linear interpolation.

55. The system according to any one of Clauses 39 to 54, wherein the processor is configured to calculate an input quality score indicating an extent to which each data point collected from the secondary sample is related to the data points within the reference dataset.

56. The system according to Clause 55, wherein calculating the input quality score comprises obtaining a normalized average distance of each data point collected from the secondary sample to the k nearest neighbors.

57. The system according to any one of Clauses 39 to 56, wherein the processor is configured to calculate an output quality score measuring the separation of the k nearest neighbors in the dimensionally reduced first dataset.

58. The system according to Clause 57, wherein calculating the output quality score comprises obtaining a normalized average distance of each of the k nearest neighbors within the dimensionally reduced reference dataset.

59. The system according to any one of Clauses 33 to 58, wherein the processor is in a data-receiving relationship with a flow cytometer, and the reference and secondary datasets are comprised of flow cytometer data.

60. The system according to any one of Clauses 33 to 59, wherein the reference dataset is a uniformly down-sampled reference dataset or a density-based down-sampled reference dataset.

61. The system according to any one of Clauses 33 to 60, wherein the processor is configured to:
receive the reference dataset; and
perform the first dimensionality reduction.

62. The system according to any one of Clauses 33 to 61, wherein the processor is configured to transform the reference dataset and the secondary dataset.

63. The system according to Clause 62, wherein the processor is configured to transform parameters in the reference dataset and parameters in the secondary dataset with a linear function, logarithmic function, a hyperbolic arcsine function, or a biexponential function.

64. The system according to any one of Clauses 33 to 63, wherein the processor is configured to calculate a dimensionality reduction for a plurality of secondary datasets comprising data points collected from a plurality of secondary samples.

65. A non-transitory computer readable storage medium comprising instructions stored thereon for dimensionality reduction by a method comprising:
receiving a secondary dataset comprising data points collected from a secondary sample; and
calculating a second dimensionality reduction for the secondary dataset based on a first dimensionality reduction of a reference dataset comprising data points collected from a reference sample, wherein the second dimensionality reduction is compatible with the dimensionally reduced reference dataset.

66. The non-transitory computer readable storage medium according to Clause 65, wherein calculating the second dimensionality reduction comprises determining data points collected from the reference sample that are closest to each data point collected from the secondary sample with respect to a given data parameter.

67. The non-transitory computer readable storage medium according to Clause 66, wherein calculating the second dimensionality reduction comprises calculating k nearest neighbors within the reference dataset for each data point collected from the secondary sample.

68. The non-transitory computer readable storage medium according to Clause 67, wherein k ranges from 1 to m, where m is half of the number of data points in the reference sample.

69. The non-transitory computer readable storage medium according to Clause 68, wherein k ranges from 2 to 5.

70. The non-transitory computer readable storage medium according to any one of Clauses 67 to 69, wherein the method comprises calculating the k nearest neighbors using a vantage-point tree, a k-dimensional tree, ball tree, cover tree, locality-sensitive hashing, hierarchical navigable small world, approximate nearest neighbors with random projection trees, GPU-based KNN search, or a brute force KNN search, or a brute force KNN search.

71. The non-transitory computer readable storage medium according to any one of Clauses 67 to 70, wherein the method comprises calculating a distance between each data point collected from the secondary sample and each nearest neighbor of the k nearest neighbors.

72. The non-transitory computer readable storage medium according to Clause 71, wherein the distance is a Manhattan distance.

73. The non-transitory computer readable storage medium according to Clause 71, wherein the distance is a Euclidean distance.

74. The non-transitory computer readable storage medium according to Clause 71, wherein the distance is a Chebyshev distance.

75. The non-transitory computer readable storage medium according to Clause 71, wherein the distance is a Minkowski distance.

76. The non-transitory computer readable storage medium according to Clause 71, wherein the distance is a cosine distance.

77. The non-transitory computer readable storage medium according to any one of Clauses 71 to 76, wherein the method comprises calculating the second dimensionality reduction based on a weighted average of positions of the k nearest neighbors.

78. The non-transitory computer readable storage medium according to Clause 77, wherein the method further comprises calculating the weighted average using a weight obtained for each nearest neighbor of the k nearest neighbors based on the distance.

79. The non-transitory computer readable storage medium according to Clause 78, wherein the processor is configured to obtain the weight as follows:

$${w}_{i} = \frac{1 + a}{1 + {d}_{i} + b}$$

wherein:
wᵢ is the weight;
dᵢ is the distance; and
a and b are real numbers.

80. The non-transitory computer readable storage medium according to Clause 78, wherein the method comprises obtaining the weight as follows:

$${w}_{i} = f ( \frac{1 + a}{1 + {d}_{i} + b} )$$

wherein:
f is any monotonic entirely non-decreasing function;
wᵢ is the weight;
dᵢ is the distance; and
a and b are real numbers.

81. The non-transitory computer readable storage medium according to Clause 78, wherein the method comprises obtaining the weight as follows:

$${w}_{i} = \frac{1}{1 + {d}_{i}}$$

wherein:
wᵢ is the weight; and
dᵢ is the distance.

82. The non-transitory computer readable storage medium according to any one of Clauses 65 to 81, wherein the secondary dataset comprises data points associated with a plurality of parameters that are matched to the reference dataset.

83. The non-transitory computer readable storage medium according to Clause 82, wherein the method further comprises creating derived parameters for the secondary dataset based parameters of the dimensionally reduced reference dataset.

84. The non-transitory computer readable storage medium according to Clause 83, wherein the derived parameters are fluorescence parameters, scatter parameters, imaging parameters or categorical parameters.

85. The non-transitory computer readable storage medium according to Clause 89, wherein the derived parameters are fluorescence parameters.

86. The non-transitory computer readable storage medium according to any one of Clauses 83 to 85, wherein creating the derived parameters for the secondary dataset comprises linear interpolation.

87. The non-transitory computer readable storage medium according to any one of Clauses 71 to 86, wherein the method further comprises calculating an input quality score indicating an extent to which each data point collected from the secondary sample is related to the data points within the reference dataset.

88. The non-transitory computer readable storage medium according to Clause 87, wherein calculating the input quality score comprises obtaining a normalized average distance of each data point collected from the secondary sample to the k nearest neighbors.

89. The non-transitory computer readable storage medium according to any one of Clauses 71 to 88, wherein the method further comprises calculating an output quality score measuring the separation of the k nearest neighbors in the dimensionally reduced first dataset.

90. The non-transitory computer readable storage medium according to Clause 89, wherein calculating the output quality score comprises obtaining a normalized average distance of each of the k nearest neighbors within the dimensionally reduced reference dataset.

91. The non-transitory computer readable storage medium according to any one of Clauses 65 to 90, wherein the reference and secondary datasets are comprised of flow cytometer data.

92. The non-transitory computer readable storage medium according to any one of Clauses 65 to 91, wherein the reference dataset is a uniformly down-sampled reference dataset or a density-based down-sampled dataset.

93. The computer-implemented method according to any one of Clauses 65 to 92, wherein the method comprises:
receiving the reference dataset; and
performing the first dimensionality reduction.

94. The non-transitory computer readable storage medium according to any one of Clauses 65 to 93, wherein the method further comprises transforming the reference dataset and the secondary dataset.

95. The non-transitory computer readable storage medium according to Clause 94, wherein the method comprises transforming parameters in the reference dataset and parameters in the secondary dataset with a linear function, logarithmic function, a hyperbolic arcsine function, or a biexponential function.

96. The non-transitory computer readable storage medium according to any one of Clauses 65 to 95, wherein the method comprises calculating a dimensionality reduction for a plurality of secondary datasets comprising data points collected from a plurality of secondary samples.

The following is presented by way of example and not by way of limitation.

### Experimental

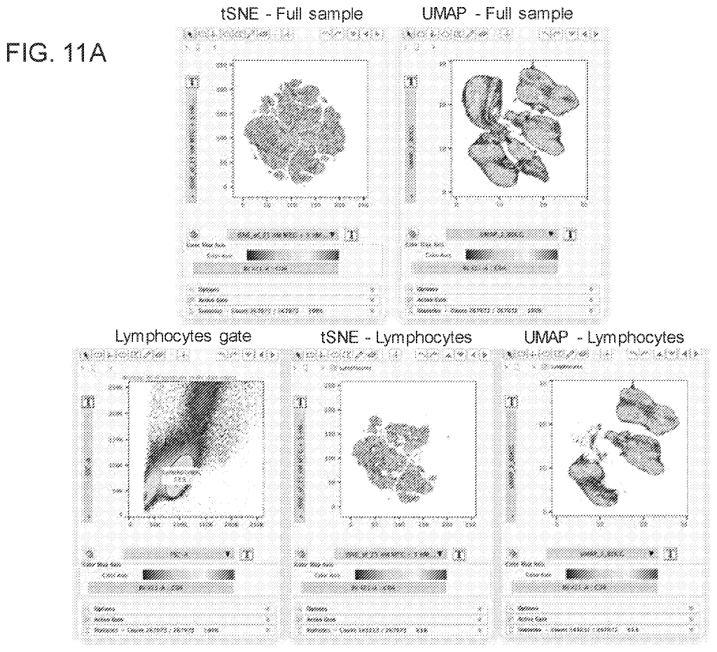

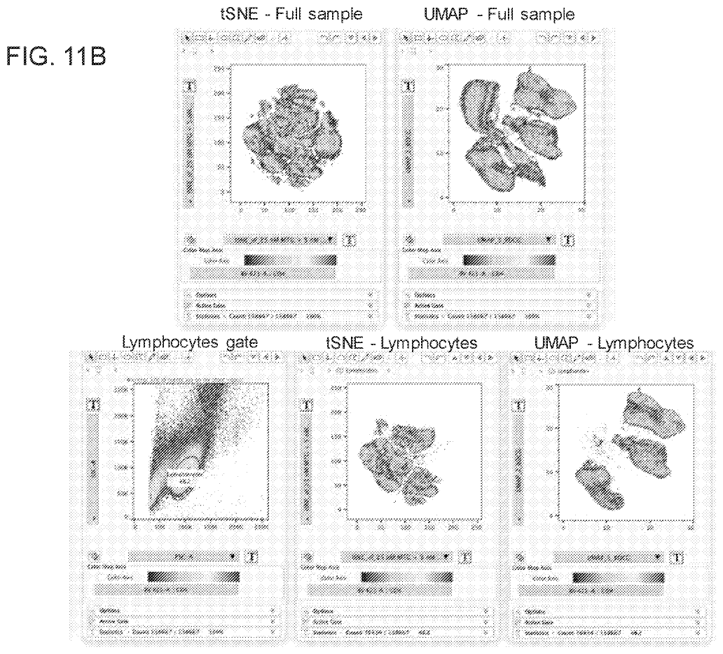

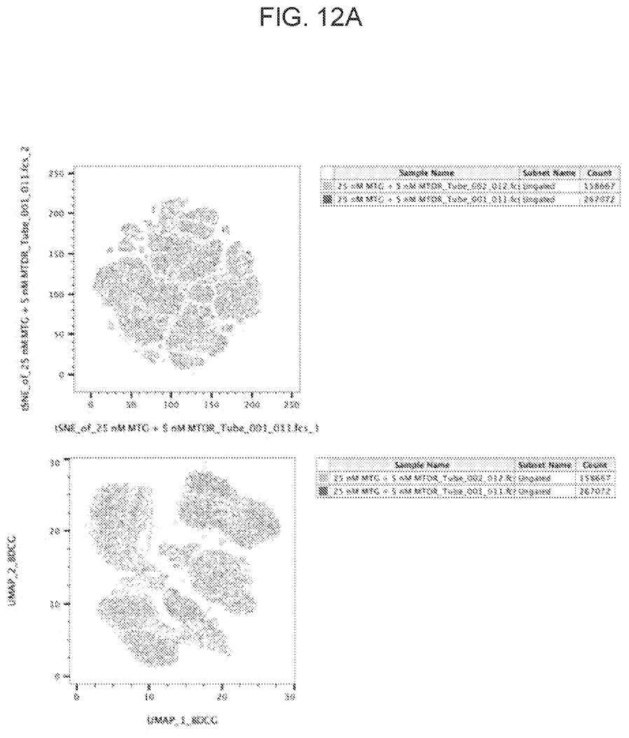

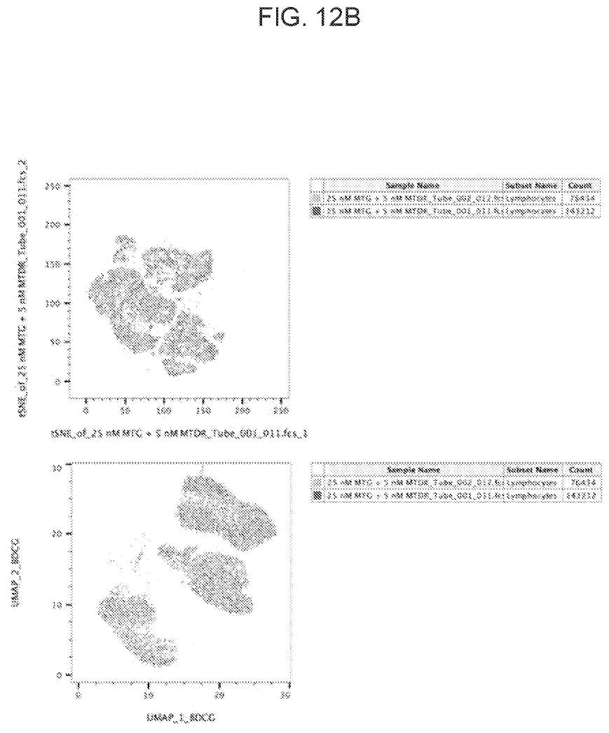

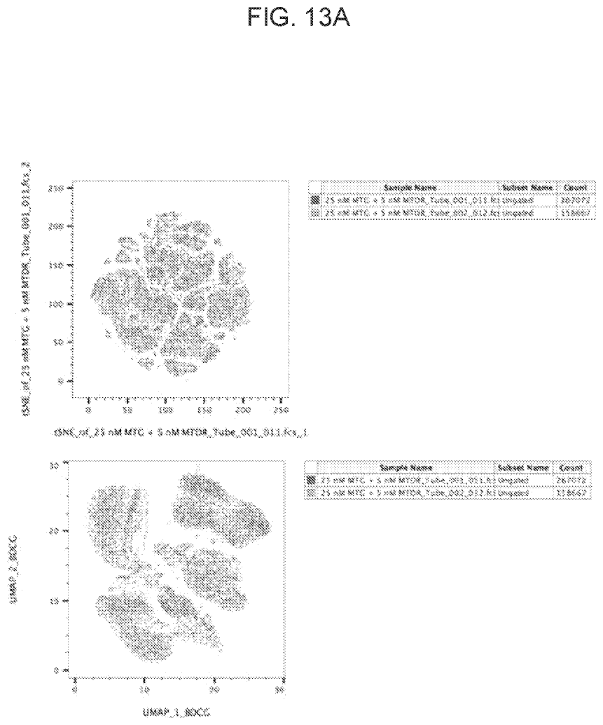

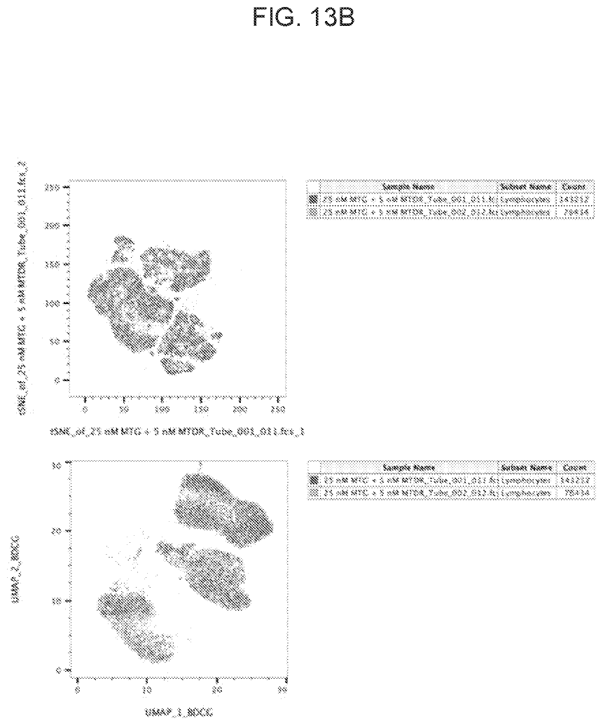

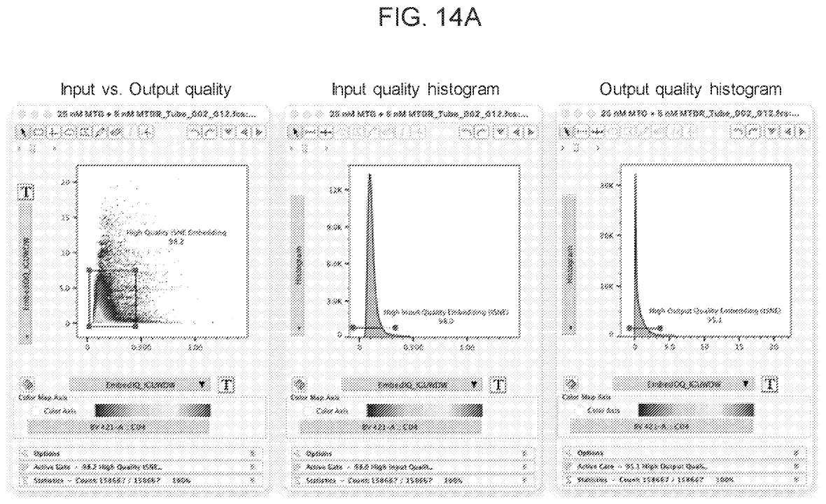

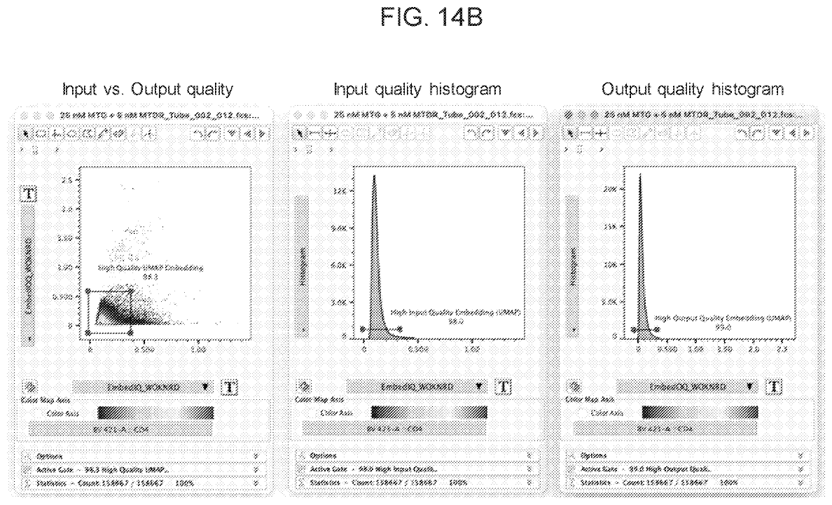

Flow cytometer data for a first sample (sample S1) and a second sample (sample S2) were obtained from public FlowRepository dataset FR-FCM-ZYX4. tSNE and UMAP were calculated on sample S1 (FIG. 11A), and compatible tSNE and UMAP were derived using the algorithm described in FIGS. 2-3 for sample S2 (FIG. 11B). A lymphocytes gate on FS/SS followed by inspection of the lymphocytes population in the tSNE and UMAP space was carried out on both samples as a “sanity check”. Plots illustrating matching embeddings using overlays are shown in FIG. 12A-12B. Plots illustrating matching embeddings using overlays having a switched sample order are shown in FIG. 13A-13B. Input and output quality scores were also calculated for tSNE (FIG. 14A) and UMAP (FIG. 14B). As shown in FIG. 14A-14B, input and output quality score can be leveraged to gate events with high quality embedding results. These scores allow users to focus further downstream analysis on data with high quality embedding results, i.e., on events where the derived embedding represents a true approximation of an ideal position of those events in the compatible embedding. Specifically, there are two main reasons for why this may not be the case for certain events: (i) a similar event population has not been present in the reference sample S1, thus the algorithm does not have sufficient data to place such events in the compatible embedding, or (ii) a similar event population from a reference sample S1 is not placed consistently in the dimensionality reduced embedding. The former will be indicated by a bad input quality score for such events, the latter will be indicated by a bad output score for such events. In addition, the output quality score may be used as a quality indicator for the embedding performed on the reference sample S1 using any of the state-of-the-art dimensionality reductions techniques. Specifically, a bad output quality score for a large proportion of events may indicate that the dimensionality reduction performed on the reference sample S1 using such state-of-the-art dimensionality reductions techniques isn't a faithful representation of the data with respect to maintaining what's typically referenced to as the local structure of the data, i.e., events that are close to each other R are not close to each other in D.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1-32. (canceled)

33. A system comprising a processor comprising memory operably coupled to the processor, wherein the memory comprises instructions stored thereon, which when executed by the processor, cause the processor to:
receive a secondary dataset comprising data points collected from a secondary sample; and
calculate a second dimensionality reduction for the secondary dataset based on a first dimensionality reduction of a reference dataset comprising data points collected from a reference sample, wherein the second dimensionality reduction is compatible with the dimensionally reduced reference dataset.

34. The system according to claim 33, wherein calculating the second dimensionality reduction comprises determining data points collected from the reference sample that are closest to each data point collected from the secondary sample with respect to given data parameters.

35. The system according to claim 34, wherein calculating the second dimensionality reduction comprises calculating k nearest neighbors within the reference dataset for each data point collected from the secondary sample.

36. The system according to claim 35, wherein k ranges from 1 to m, where m is half of the number of data points in the reference sample.

37. (canceled)

38. The system according to claim 35, wherein the processor is configured to calculate the k nearest neighbors using a vantage-point tree, a k-dimensional tree, ball tree, cover tree, locality-sensitive hashing, hierarchical navigable small world, approximate nearest neighbors with random projection trees, GPU-based KNN search, or a brute force KNN search.

39. The system according to claim 35, wherein the processor is configured to calculate a distance between each data point collected from the secondary sample and each nearest neighbor of the k nearest neighbors.

40-44. (canceled)

45. The system according to claim 39, wherein the processor is configured to calculate the second dimensionality reduction based on a weighted average of positions of the k nearest neighbors.

46. The system according to claim 45, wherein the processor is configured to calculate the weighted average using a weight obtained for each nearest neighbor of the k nearest neighbors based on the distance.

47-49. (canceled)

50. The system according to claim 33, wherein the secondary dataset comprises data points associated with a plurality of parameters that are matched to the reference dataset.

51. The system according to claim 50, wherein the processor is configured to create derived parameters for the secondary dataset based parameters of the dimensionally reduced reference dataset.

52. The system according to claim 51, wherein the derived parameters are fluorescence parameters, scatter parameters, imaging parameters or categorical parameters.

53. The system according to claim 52, wherein the derived parameters are fluorescence parameters.

54. The system according to claim 51, wherein
creating the derived parameters for the secondary dataset comprises linear interpolation.

55. The system according to claim 39, wherein the processor is configured to calculate an input quality score indicating an extent to which each data point collected from the secondary sample is related to the data points within the reference dataset.

56. (canceled)

57. The system according to claim 39, wherein the processor is configured to calculate an output quality score measuring the separation of the k nearest neighbors in the dimensionally reduced first dataset.

58. (canceled)

59. (canceled)

60. (canceled)

61. (canceled)

62. (canceled)

63. (canceled)

64. (canceled)

65-96. (canceled)
